(19) United States
(12) Patent Application Publication
(10) Pub. No.: US 2012/0101163 A1
(43) Pub. Date: Apr. 26, 2012

(54) COMPOSITION FOR INHIBITING CANCER METASTASIS

(75) Inventors: Wen-Chi Hou, Taipei (TW); Yin-Shiou Lin, Taipei (TW); Wen-Chung Wu, Taipei (TW)

(73) Assignee: TAIPEI MEDICAL UNIVERSITY, Taipei (TW)

(21) Appl. No.: 13/206,495

(22) Filed: Aug. 9, 2011

(30) Foreign Application Priority Data
Oct. 25, 2010 (TW) 099136263

(51) Int. Cl. A61K 31/195; A61P 35/00; A61K 31/16

(52) U.S. Cl. 514/563; 514/616

(57) ABSTRACT

The invention provides a composition for inhibiting cancer metastasis, including: an effective amount of an amino acid hydroxamic acid derivative having a formula as shown as formula (I), formula (II) or formula (III):

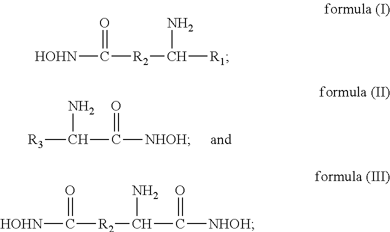

and a pharmaceutically acceptable carrier or salt, wherein the amino acid hydroxamic acid derivative has an effect for inhibiting cancer metastasis.

### CROSS REFERENCE TO RELATED APPLICATIONS

This application claims priority of Taiwan Patent Application No. 099136263, filed on Oct. 25, 2010, the entirety of which is incorporated by reference herein.

### INCORPORATION BY REFERENCE OF SEQUENCE LISTING

A sequence listing submitted as a text file via EFS-Web is incorporated herein by reference. The text file containing the sequence listing is named “9049-A52019-US_Seq_Listing.txt”; its date of creation is Mar. 21, 2011; and its size is 1,370 bytes.

### BACKGROUND OF THE INVENTION

1. Field of the Invention

The present invention relates to a composition for inhibiting cancer metastasis, and in particular relates to a composition for inhibiting cancer metastasis which contains an amino acid hydroxamic acid derivative as an active ingredient.

2. Description of the Related Art

“Cancer metastasis” is a cancer cell process, wherein a cancer cell departs from a carcinoma in situ and travels elsewhere by the circulatory system to form a tumor in a different location from its original (Woodhouse et al., 1997; Okada et al., 1998). Cancer metastasis is a complicated process comprising a series of interactions between cancer cells and their surrounding environments (Huang et al., 2005; Itoh et al., 2005; Lee et al., 2006).

Cancer cells proliferate due to external factors and secretions therefrom, may stimulate angiogenesis during the proliferation process. When a carcinoma in situ grows to a certain level, some cancer cells will depart therefrom, and migrate and invade surrounding tissues. Following, the departed cancer cells may adhere to and penetrate the wall of nearby blood vessels or lymph vessels, wherein they may travel with the blood or lymph, through circulation, to another position of a body (Lirdprapamongkol et al., 2005). When cancer cells, surviving in the circulatory system, arrive at an appropriate environment, they will adhere to the walls of the blood vessel or lymph vessel nearby and penetrate them to grow in surrounding tissues. Thus, the cancer cells will have migrated to another position of a body (Okada et al., 1998; Ala-aho et al., 2005).

Some important mechanisms during the cancer metastasis process comprise: desorption between cancer cells, adhesion of cancer cells and other cells (Lee et al., 2005; Ouyang et al., 2005; Zhang et al., 2005), secretion of enzymes by cancer cells (such as matrix metalloproteinase (MMP)), decomposition of extracellular matrix (ECM), and angiogenesis at cancered positions (Ahmad et al., 1997; Woodhouse et al., 1997; Ala-aho et al., 2005), etc. Accordingly, for future research and development of anti-cancer drugs, these mechanisms may be targeted (Ahmad et al., 1997; Woodhouse et al., 1997).

Matrix metalloproteinases (MMPs), are a group of proteases containing zinc, which are isolated from a tail of a tadpole (Gross and Lapiere, 1962).

In physiology, matrix metalloproteinases participate in embryo progression, ovulation, repairing for injured cells or tissues, and regeneration of blood vessels, etc. While in pathology, matrix metalloproteinases participate in angiogenesis, regeneration of blood vessels, and growth, metastasis, migration and invasion of cancer cells, cardiovascular diseases, arteriosclerosis, lung diseases, inflammation, and arthritis, etc. (John and Tuszynski, 2001; Chakraborti et al., 2003; Folgueras et al., 2004; Ala-aho et al., 2005; Bjorklund and Koivunen, 2005). Cancer cells having invasive abilities will release cell lysis enzymes during the metastasis process, such as lysosomal hydrolase and matrix metalloproteinases. Matrix metalloproteinases are able to lyse the extracellular matrix and basement membranes to infiltrate cancer cells into the blood or lymph system (Alicia et al., 2004). Lysis of the extracellular matrix is closely associated with growth, invasion and metastasis of malignant tumors, and thus matrix metalloproteinases possess a very important place therein (Chakraborti et al., 2003). Therefore, the lysis ability of the matrix metalloproteinase and invasive ability of cancer cells, present a positive correlation (Ahmad et al., 1997; Ala-aho et al., 2005). Resent research has indicated that since the extracellular matrix and basement membrane mainly consist of type IV collagen, and type IV collagenases are capable of decomposing type IV collagen, and thus among the matrix metalloproteinases, type IV collagenases (matrix metalloproteinase-2 and matrix metalloproteinase-9) is mostly related with metastasis of tumor cells and angiogenesis (Lambert et al., 1997; John and Tuszynski, 2001; Hwang et al., 2006). It has been found that active states for matrix metalloproteinase-2 and matrix metalloproteinase-9 at metastasis positions of many patients were higher than that of normal people (Huang et al., 2005). Therefore, inhibition of matrix metalloproteinase-2 and matrix metalloproteinase-9 activities may indicate a very important area for development of anti-metastasis drugs (Ala-aho et al., 2005; Huang et al., 2005).

Regeneration of blood vessels possess a physiological function of wound repair and growth in human bodies. Previous research has found that the process of metastasis will accompany angiogenesis. Cancer cells and tissues nearby may secrete angiogenic molecules, such as vascular endothelial growth factors, (VEGF) and basic fibroblast growth factors (bFGF), wherein the process of angiogenesis is described in the following. After stimulated and activated by angiogenic molecules, the vascular endothelial cells secrete enzymes decomposing and destroying the connective tissues near the cancer cells and proliferating the endothelial cells, wherein the proliferated endothelial cells move toward the cancer cells or tissues secreting angiogenic molecules. Following, the endothelial cells recombine to form a regenerated blood vessel. The cancer cells move through the circulatory system by the regenerated blood vessels and metastasize to other organs. Thus, the regenerated blood vessel may raise nutrient assimilation for the cancer cells (J. Folkman, 1995; Peter Carmeliet and Rakesh K. Jain, 2000).

90% of death for cancer patients are due to cancer cell metastasis (Elvin et al., 2005). Clinically, 30% of carcinoma in situ patients were found to have the appearance of metastasis, and 70% of the patients were found to have the appearance of metastasis during the progress of tumor development. Also, note that even if tumors are excised, remaining cancer cells may still endanger the life of a cancerous patient (John and Tuszynski, 2001). Accordingly, new therapies for metastasis provide help for improving the survival rate of cancer patients (Ahmad et al., 1997; Woodhouse et al., 1997; John and Tuszynski, 2001).

### BRIEF SUMMARY OF THE INVENTION

The invention provides a composition for inhibiting cancer metastasis, comprising: an effective amount of an amino acid hydroxamic acid derivative having a formula as shown as formula (I), formula (II) or formula (III):

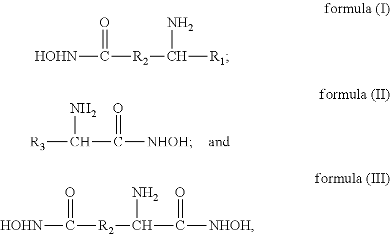

wherein R₁ comprises carboxyl, C₁-C₆ alkyl, C₁-C₆ alkoxy, NO, or NHOH, R₂ comprises C₁-C₆ alkyl, C₁-C₆ alkoxy or phenyl, and R₃ comprises the side chain of tryptophan, the side chain of valine, the side chain of isoleucine, the side chain of threonine, the side chain of lysine, the side chain of phenylalanine, the side chain of leucine, the side chain of methionine, the side chain of histidine, the side chain of glycine, the side chain of glutamic acid, the side chain of hydroxyproline, the side chain of alanine, the side chain of serine, the side chain of glutamine, the side chain of cystine, the side chain of proline, the side chain of aspartic acid, the side chain of citrulline, the side chain of arginine, C₁-C₆ alkyl, C₁-C₆ alkoxy, NO or NHOH; and a pharmaceutically acceptable carrier or salt, wherein the amino acid hydroxamic acid derivative has an effect for inhibiting cancer metastasis.

A detailed description is given in the following embodiments with reference to the accompanying drawings.

### BRIEF DESCRIPTION OF THE DRAWINGS

The present invention can be more fully understood by reading the subsequent detailed description and examples with references made to the accompanying drawings, wherein:

FIG. 1 shows the viability of the HT 1080 cells after being treated with the 8 kinds of the amino acid hydroxamic acid derivatives for 24 hours, respectively;

FIGS. 2 (A) and (B) show effects of the 8 kinds of the amino acid hydroxamic acid derivatives on the matrix metalloproteinase-2 and matrix metalloproteinase-9 of the HT 1080 cells after the HT 1080 cells were treated with the 8 kinds of the amino acid hydroxamic acid derivatives for 24 hours, respectively; FIG. 2 (A) shows the electrophoresis results for the equal amounts of the cellular proteins of the HT 1080 cells treated with the different amino acid hydroxamic acid derivatives, respectively. FIG. 2 (B) shows the relative activities of the matrix metalloproteinase-2 and matrix metalloproteinase-9 of the HT 1080 cells treated with the different amino acid hydroxamic acid derivatives, respectively;

FIG. 3 shows the viability of the HT 1080 cells after being treated withdifferent concentrations of _{L}-Glutamic acid γ-hydroxamate (DH) for 24 hours, respectively;

FIGS. 4 (A) and (B) show effects of different concentrations of _{L}-Glutamic acid γ-hydroxamate (DH) and different concentrations of _{L}-Glutamic acid (D) on the matrix metalloproteinase-2 and matrix metalloproteinase-9 of the HT 1080 cells, respectively, after the HT 1080 cells were treated with the different concentrations of _{L}-Glutamic acid γ-hydroxamate (DH) and different concentrations of _{L}-Glutamic acid (D) for 24 hours, respectively; FIG. 4 (A) shows the electrophoresis results for the equal amounts of the cellular proteins of the HT 1080 cells treated with the different concentrations of _{L}-Glutamic acid γ-hydroxamate (DH) and different concentrations of _{L}-Glutamic acid (D), respectively; FIG. 4 (B) shows the relative activities of the matrix metalloproteinase-2 and matrix metalloproteinase-9 of the HT 1080 cells treated with the different concentrations of _{L}-Glutamic acid γ-hydroxamate (DH) and different concentrations of _{L}-Glutamic acid (D), respectively;

FIGS. 5 (A) and (B) show effects of different concentrations of _{L}-Glutamic acid γ-hydroxamate (DH) on the matrix metalloproteinase-2 and matrix metalloproteinase-9 secreted in the medium by the HT 1080 cells after the cultured medium of the HT 1080 cells induced by PMA were treated with the different concentrations of _{L}-Glutamic acid γ-hydroxamate (DH) for 24 hours, respectively; FIG. 5 (A) shows the electrophoresis results for the equal amounts of cultured medium of the HT 1080 cells treated with the different concentrations of _{L}-Glutamic acid γ-hydroxamate (DH); FIG. 5 (B) shows the relative activities of the matrix metalloproteinase-2 and matrix metalloproteinase-9 secreted in the medium by the HT 1080 cells treated with the different concentrations of _{L}-Glutamic acid γ-hydroxamate (DH);

FIG. 6 (A) and FIGS. 6 (B) and (C) show effects of different concentrations of _{L}-Glutamic acid γ-hydroxamate (DH) on the protein expressions and protein activities of the matrix metalloproteinase-2 and matrix metalloproteinase-9 in the HT 1080 cells and secreted in the medium by the HT 1080 cells after the cultured medium of the HT 1080 cells were treated with the different concentrations of _{L}-Glutamic acid γ-hydroxamate (DH) for 24 hours, respectively; FIG. 6 (A) shows protein expression amounts of the matrix metalloproteinase-2 and matrix metalloproteinase-9 in the HT 1080 cells and secreted in the medium by the HT 1080 cells, respectively; FIGS. 6 (B) and (C) show the protein activities of the matrix metalloproteinase-2 and matrix metalloproteinase-9 in the HT 1080 cells and secreted in the medium by the HT 1080 cells, respectively.

FIGS. 7 (A) and (B) show effects of different concentrations of _{L}-Glutamic acid γ-hydroxamate (DH) on the gene expressions of the matrix metalloproteinase-2 and matrix metalloproteinase-9 of the HT 1080 cells, respectively, after the HT 1080 cells were treated with the different concentrations of _{L}-Glutamic acid γ-hydroxamate (DH) for 6 hours, respectively; FIG. 7 (A) shows the gene expression results analyzed by RT PCR for the matrix metalloproteinase-2 and matrix metalloproteinase-9 of the HT 1080 cells treated with the different concentrations of _{L}-Glutamic acid γ-hydroxamate (DH), respectively. FIG. 7 (B) shows the relative gene expression amounts of the matrix metalloproteinase-2 and matrix metalloproteinase-9 of the HT 1080 cells treated with the different concentrations of L-Glutamic acid γ-hydroxamate (DH), respectively.

FIGS. 8 (A) and (B) show effects of different concentrations of _{L}-Glutamic acid γ-hydroxamate (DH) on the migration ability of the HT 1080 cells. FIG. 8 (A) shows wound healing assay results for the HT 1080 cells treated with the different concentrations of _{L}-Glutamic acid γ-hydroxamate (DH); FIG. 8 (B) shows the cell migration rates for the HT 1080 cells treated with the different concentrations of _{L}-Glutamic acid γ-hydroxamate (DH);

FIGS. 9 (A) and (B) show effects of different concentrations of _{L}-Glutamic acid γ-hydroxamate (DH) on the invasion ability of the HT 1080 cells. FIG. 9 (A) shows in the invasion assay, the HT 1080 cells which moved to the lower chambers of the transwells after the HT 1080 cells were treated with the different concentrations of _{L}-Glutamic acid γ-hydroxamate (DH), respectively; FIG. 9 (B) shows the invasion rates for the HT 1080 cells treated with the different concentrations of _{L}-Glutamic acid γ-hydroxamate (DH), respectively;

FIGS. 10 (A) and (B) show effects of different concentrations of _{L}-Glutamic acid γ-hydroxamate (DH) on HT 1080 cell adhesion ability. FIG. 10 (A) shows cell adhesion test results for the HT 1080 cells treated with the different concentrations of _{L}-Glutamic acid γ-hydroxamate (DH), respectively; FIG. 10 (B) shows the adhesion rates for the HT 1080 cells treated with the different concentrations of _{L}-Glutamic acid γ-hydroxamate (DH), respectively;

FIGS. 11 (A), (B) and (C) show effects of _{L}-Glutamic acid γ-hydroxamate (DH) on the lung metastasis and the viability of mice. FIG. 11 (A) show the lung metastasis conditions of the mice in the control group (no cancer induced), the blank group (B16-F10 induced without a drug being administered) and the drug administered group (B16-F10 induced with _{L}-Glutamic acid γ-hydroxamate (DH) administered). FIG. 11 (B) show the number of lung metastasis and viability of the mice in the control group (no cancer induced), in the blank group (B16-F10 induced without a drug being administered) and in the drug administered group (B16-F10 induced with _{L}-Glutamic acid γ-hydroxamate (DH) administered), respectively. FIG. 11 (C) show the lung weights of the mice in the control group (no cancer induced), in the blank group (B16-F10 induced without a drug being administered) and in the drug administered group (B16-F10 induced with _{L}-Glutamic acid γ-hydroxamate (DH) administered), respectively;

FIGS. 12 (A) and (B) show effects of _{L}-Glutamic acid γ-hydroxamate (DH) on the appearances and weights of the mice induced by B16-F10 cells; FIG. 12 (A) show the appearances of the mice in the control group (no cancer induced), in the blank group (B 16-F10 cells induced without a drug being administered) and in the drug administered group (B16-F10 induced with _{L}-Glutamic acid γ-hydroxamate (DH) administered), respectively; FIG. 12 (B) show the weight changes of the mice in the blank group (B16-F10 cells induced without a drug being administered) and in the drug administered group (B16-F10 induced with _{L}-Glutamic acid γ-hydroxamate (DH) administered), respectively.

FIGS. 13 (A) and (B) show effects of _{L}-Aspartic acid β-hydroxamate (CH) on the viability and angiogenesis for the human umbilical vein endothelial cells. FIG. 13 (A) shows the viability of the human umbilical vein endothelial cells after being treated with the different concentrations of _{L}-Aspartic acid β-hydroxamate (CH) for 24 hours, respectively; FIG. 13 (B) shows the microscope observing results for the human umbilical vein endothelial cells treated with the different concentrations of _{L}-Aspartic acid β-hydroxamate (CH), respectively;

FIGS. 14 (A) and (B) show effects of _{L}-Glutamic acid γ-hydroxamate (DH) on the viability and angiogenesis for the human umbilical vein endothelial cells. FIG. 14 (A) shows the viability of the human umbilical vein endothelial cells after being treated with the different concentrations of _{L}-Glutamic acid γ-hydroxamate (DH) for 24 hours, respectively. FIG. 14 (B) shows the microscope observing results for the human umbilical vein endothelial cells treated with the different concentrations of _{L}-Glutamic acid γ-hydroxamate (DH), respectively.

### DETAILED DESCRIPTION OF THE INVENTION

The following description is of the best-contemplated mode of carrying out the invention. This description is made for the purpose of illustrating the general principles of the invention and should not be taken in a limiting sense. The scope of the invention is best determined by reference to the appended claims.

In one aspect of the invention, the invention uses an amino acid hydroxamic acid derivative composition as an anti-cancer metastasis drug which is able to inhibit cancer metastasis.

The invention provides a composition for inhibiting cancer metastasis using an amino acid hydroxamic acid derivative as an active ingredient. In one embodiment, the composition may comprise an effective amount of an amino acid hydroxamic acid derivative and a pharmaceutically acceptable carrier or salt, wherein a formula of the amino acid hydroxamic acid derivative is as shown as formula (I), formula (II) or formula (III):

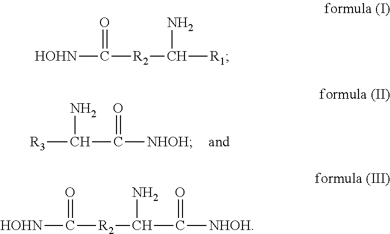

In the above-mentioned formula (I), formula (II) or formula (III), R₁ may comprise carboxyl, C₁-C₆ alkyl, C₁-C₆ alkoxy, NO, or NHOH, R₂ may comprise C₁-C₆ alkyl, C₁-C₆ alkoxy or phenyl, and R₃ may comprise the side chain of tryptophan, the side chain of valine, the side chain of isoleucine, the side chain of threonine, the side chain of lysine, the side chain of phenylalanine, the side chain of leucine, the side chain of methionine, the side chain of histidine, the side chain of glycine, the side chain of glutamic acid, the side chain of hydroxy proline, the side chain of alanine, the side chain of serine, the side chain of glutamine, the side chain of cystine, the side chain of proline, the side chain of aspartic acid, the side chain of citrulline, the side chain of arginine, C₁-C₆ alkyl, C₁-C₆ alkoxy, NO or NHOH.

In one embodiment, a content of the amino acid hydroxamic acid derivative in the above-mentioned composition is about 0.01-50 wt %.

The above-mentioned amino acid hydroxamic acid derivative may have an effect for inhibiting cancer metastasis. The type of cancer metastasis which may be inhibited by the above-mentioned amino acid hydroxamic acid derivative may comprise, but is not limited to, lung cancer, breast cancer, leukaemia, etc. In one embodiment, the effect for inhibiting cancer metastasis may comprise inhibiting cancer cell migration, inhibiting cancer cell invasion, inhibiting cancer cell adhesion and/or inhibiting angiogenesis. The cancer cell may comprise, but is not limited to, a fibrosarcoma cell, a melanoma cell, a breast cancer cell and a lung cancer cell, etc.

The foregoing amino acid hydroxamic acid derivative at a concentration without cytotoxicity may inhibit one of the cancer metastasis biomarkers, matrix metalloproteinase-2 and/or matrix metalloproteinase-9. In one embodiment, the amino acid hydroxamic acid derivative is capable of inhibiting gene and protein expressions of matrix metalloproteinase-2 and matrix metalloproteinase-9 of a cancer cell.

The foregoing amino acid hydroxamic acid derivative may inhibit protein expression amount of metalloproteinase-2 and/or matrix metalloproteinase-9 through inhibiting gene transcriptional expression (mRNA) of metalloproteinase-2 and/or matrix metalloproteinase-9, and thus further inhibit protein activity of metalloproteinase-2 and/or matrix metalloproteinase-9.

In one embodiment, for an about 0.1-2 mM concentration of the amino acid hydroxamic acid derivative, the inhibition rate to the gene transcriptional expression of metalloproteinase-2 and/or matrix metalloproteinase-9 of a cancer cell is about 20-80%. In another embodiment, for an about 0.1-2 mM concentration of the amino acid hydroxamic acid derivative, the inhibition rate to the protein expression amount of metalloproteinase-2 and/or matrix metalloproteinase-9 of a cancer cell is about 40-90%. In further another embodiment, for an about 0.1-2 mM concentration of the amino acid hydroxamic acid derivative, the inhibition rate to the protein activity of metalloproteinase-2 and/or matrix metalloproteinase-9 of a cancer cell is about 40-70%.

Moreover, in a wound healing assay and colony dispersion assay, the foregoing amino acid hydroxamic acid derivative at a concentration without cytotoxicity may inhibit cancer cell migration. In one embodiment, for an about 0.1-3 mM concentration of the amino acid hydroxamic acid derivative, the inhibition rate to cancer cell migration is about 20-85%.

Furthermore, in a transmembrane test (invasion assay), the foregoing amino acid hydroxamic acid derivative at a concentration without cytotoxicity may inhibit cancer cell invasion. In one embodiment, for an about 0.1-3 mM concentration of the amino acid hydroxamic acid derivative, the inhibition rate to cancer cell invasion is about 40-85%.

In a cell adhesion assay for cancer cells, the foregoing amino acid hydroxamic acid derivative at a concentration without cytotoxicity is capable of inhibiting cancer cell adhesion. In one embodiment, for an about 0.1-3 mM concentration of the amino acid hydroxamic acid derivative, the inhibition rate to cancer cell adhesion is about 10-30%.

In an angiogenesis test (tube formation assay), the foregoing amino acid hydroxamic acid derivative at a concentration without cytotoxicity is capable of inhibiting angiogenesis. In one embodiment, for an about 0.1-1.5 mM concentration of the amino acid hydroxamic acid derivative, the inhibition rate to angiogenesis is about 10-90%.

In addition, the amino acid hydroxamic acid derivative may comprise an L-form or D-form amino acid hydroxamic acid derivative.

The amino acid hydroxamic acid derivative having a formula of formula (I) may comprise, but is not limited to, _{L}-Glutamic acid γ-hydroxamate (DH) or _{L}-Aspartic acid β-hydroxamate (CH). The formula of the _{L}-Glutamic acid γ-hydroxamate and the formula of _{L}-Aspartic acid β-hydroxamate are as shown as formula (IV) and formula (V), respectively:

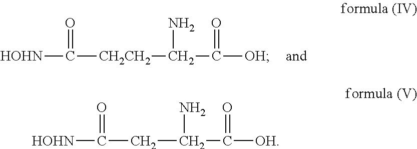

The amino acid hydroxamic acid derivative having a formula of formula (III) may comprise, but is not limited to, _{L}-Glutamic acid dihydroxamate or _{L}-Aspartic acid dihydroxamate. The formula of _{L}-Glutamic acid dihydroxamate and the formula of _{L}-Aspartic acid dihydroxamate are as shown as formula (VI) and formula (VII), respectively:

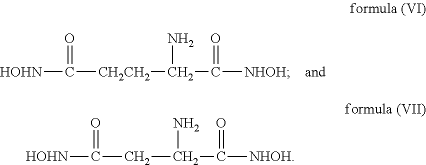

In one embodiment, the amino acid hydroxamic acid derivative having a formula of formula (I) is _{L}-Glutamic acid γ-hydroxamate (DH), and the formula thereof is as shown as formula (IV):

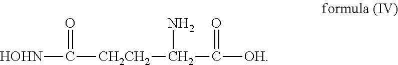

In one embodiment, the _{L}-Glutamic acid γ-hydroxamate in the above-mentioned composition is about 0.01-50 wt %.

The _{L}-Glutamic acid γ-hydroxamate (DH) may have an effect for inhibiting cancer metastasis. The cancer whose metastasis may be inhibited by the _{L}-Glutamic acid γ-hydroxamate (DH) may comprise, but is not limited to, lung cancer, breast cancer, leukaemia, etc. In one embodiment, the effect for inhibiting cancer metastasis may comprise inhibiting cancer cell migration, inhibiting cancer cell invasion, inhibiting cancer cell adhesion and/or inhibiting angiogenesis. The cancer cell may comprise, but is not limited to, a fibrosarcoma cell, a melanoma cell, a breast cancer cell and a lung cancer cell, etc.

The _{L}-Glutamic acid γ-hydroxamate (DH) at a concentration without cytotoxicity may inhibit one of the cancer metastasis biomarkers, matrix metalloproteinase-2 and/or matrix metalloproteinase-9.

The _{L}-Glutamic acid γ-hydroxamate (DH) may inhibit protein expression amount of metalloproteinase-2 and/or matrix metalloproteinase-9 through inhibiting gene transcriptional expression (mRNA) of metalloproteinase-2 and/or matrix metalloproteinase-9, and thus further inhibit protein activity of metalloproteinase-2 and/or matrix metalloproteinase-9. In one embodiment, for an about 0.1-2 mM concentration of the _{L}-Glutamic acid γ-hydroxamate (DH), the inhibition rate to the gene transcriptional expression of metalloproteinase-2 and/or matrix metalloproteinase-9 of a cancer cell is about 20-80%. In another embodiment, for an about 0.1-2 mM concentration of the _{L}-Glutamic acid γ-hydroxamate (DH), the inhibition rate to the protein expression amount of metalloproteinase-2 and/or matrix metalloproteinase-9 of a cancer cell is about 40-90%. In further another embodiment, for an about 0.1-2 mM concentration of the _{L}-Glutamic acid γ-hydroxamate (DH), the inhibition rate to the protein activity of metalloproteinase-2 and/or matrix metalloproteinase-9 of a cancer cell is about 40-70%.

Moreover, in a wound healing assay and colony dispersion assay, the _{L}-Glutamic acid γ-hydroxamate (DH) at a concentration without cytotoxicity may inhibit cancer cell migration. In one embodiment, for a.n about 0.1-3 mM concentration of the _{L}-Glutamic acid γ-hydroxamate (DH), the inhibition rate to cancer cell migration is about 20-85%.

Furthermore, in a transmembrane test (invasion assay), the _{L}-Glutamic acid γ-hydroxamate (DH) at a concentration without cytotoxicity may inhibit cancer cell invasion In one embodiment, for an about 0.1-3 mM concentration of the _{L}-Glutamic acid γ-hydroxamate (DH), the inhibition rate to cancer cell invasion is about 40-85%.

In a cell adhesion assay for cancer cells, the _{L}-Glutamic acid γ-hydroxamate (DH) at a concentration without cytotoxicity is capable of inhibiting cancer cell adhesion. In one embodiment, for an about 0.1-3 mM concentration of the _{L}-Glutamic acid γ-hydroxamate (DH), the inhibition rate to cancer cell adhesion is about 10-30%.

In an angiogenesis test (tube formation assay), the _{L}-Glutamic acid γ-hydroxamate (DH) at a concentration without cytotoxicity is capable of inhibiting angiogenesis. In one embodiment, for an about 0.1-1.5 mM concentration of the _{L}-Glutamic acid γ-hydroxamate (DH), the inhibition rate to angiogenesis is about 10-90%.

In another embodiment, the amino acid hydroxamic acid derivative having a formula of formula (I) is _{L}-Aspartic acid β-hydroxamate (CH), and the formula thereof is as shown as formula (V):

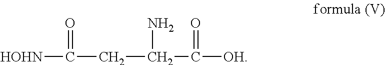

In one embodiment, the _{L}-Aspartic acid β-hydroxamate (CH) in the above-mentioned composition is about 0.01-50 wt %.

The _{L}-Aspartic acid β-hydroxamate (CH) may have an effect for inhibiting cancer metastasis. The cancer whose metastasis may be inhibited by _{L}-Aspartic acid β-hydroxamate (CH) may comprise, but is not limited to, lung cancer, breast cancer, leukaemia, etc. In one embodiment, the effect for inhibiting cancer metastasis may comprise inhibiting cancer cell migration, inhibiting cancer cell invasion, inhibiting cancer cell adhesion and/or inhibiting angiogenesis. The cancer cell may comprise, but is not limited to, a fibrosarcoma cell, a melanoma cell, a breast cancer cell and a lung cancer cell, etc.

In an angiogenesis test (tube formation assay), the _{L}-Aspartic acid β-hydroxamate (CH) at a concentration without cytotoxicity is capable of inhibiting angiogenesis. In one embodiment, for an about 0.1-1.5 mM concentration of the _{L}-Aspartic acid β-hydroxamate (CH), the inhibition rate to angiogenesis is about 10-90%.

The pharmaceutically acceptable carrier may comprise, but is not limited to, a solvent, a dispersion medium, a coating, an antibacterial and antifungal agent, or an isotonic and absorption delaying agent. The pharmaceutical composition can be formulated into dosage forms for different administrative routes utilizing conventional methods.

The pharmaceutically acceptable salt may comprise, but is not limited to, inorganic cation salts including alkali metal salts such as sodium salt, potassium salt or amine salt, alkaline-earth metal salt such as magnesium salt or calcium salt, or the salt containing bivalent or quadrivalent cation such as zinc salt, aluminum salt or zirconium salt. In addition, the pharmaceutically acceptable salt may also comprise organic salt including dicyclohexylamine salt, methyl-D-glucamine, and amino acid salt such as arginine, lysine, histidine, or glutamine.

The composition may be administered orally, parenterally by an inhalation spray or via an implanted reservoir. The parenteral method may comprise subcutaneous, intracutaneous, intravenous, intramuscular, intraarticular, intraarterial, intrasynovial, intrasternal, intrathecal, and intraleaional, as well as infusion techniques.

An oral form of the composition can comprise, but is not limited to, tablets, capsules, emulsions and aqueous suspensions, dispersions and solutions.

In another aspect of the invention, the invention may also comprise a method for inhibiting cancer metastasis. The method for inhibiting cancer metastasis comprises administering an effective amount of an amino acid hydroxamic acid derivative or an effective amount of an anti-cancer metastasis composition which contains the above-mentioned amino acid hydroxamic acid derivative and a pharmaceutically acceptable carrier or salt, to a subject in need. The appropriate acceptable carrier or salt in the composition is the same as the above-mentioned. In one embodiment, the subject may comprise a mammal, and the mammal may comprise a human.

A formula of the amino acid hydroxamic acid derivative mentioned above is as shown as formula (I), formula (II) or formula (III):

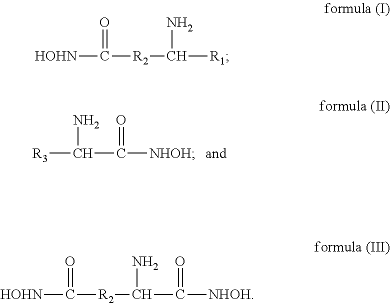

In the above-mentioned formula (I), formula (II) or formula (III), R₁ may comprise carboxyl, C₁-C₆ alkyl, C₁-C₆ alkoxy, NO, or NHOH, R₂ may comprise C₁-C₆ alkyl, C₁-C₆ alkoxy or phenyl, and R₃ may comprise the side chain of tryptophan, the side chain of valine, the side chain of isoleucine, the side chain of threonine, the side chain of lysine, the side chain of phenylalanine, the side chain of leucine, the side chain of methionine, the side chain of histidine, the side chain of glycine, the side chain of glutamic acid, the side chain of hydroxy proline, the side chain of alanine, the side chain of serine, the side chain of glutamine, the side chain of cystine, the side chain of proline, the side chain of aspartic acid, the side chain of citrulline, the side chain of arginine, C₁-C₆ alkyl, C₁-C₆ alkoxy, NO or NHOH.

The above-mentioned amino acid hydroxamic acid derivative may have an effect for inhibiting cancer metastasis. The cancer whose metastasis may be inhibited by the above-mentioned amino acid hydroxamic acid derivative may comprise, but is not limited to, lung cancer, breast cancer, leukaemia, etc. In one embodiment, the effect for inhibiting cancer metastasis may comprise inhibiting cancer cell migration, inhibiting cancer cell invasion, inhibiting cancer cell adhesion and/or inhibiting angiogenesis. The cancer cell may comprise, but is not limited to, a fibrosarcoma cell, a melanoma cell, a breast cancer cell and a lung cancer cell, etc.

The foregoing amino acid hydroxamic acid derivative at a concentration without cytotoxicity may inhibit one of the cancer metastasis biomarkers, matrix metalloproteinase-2 and/or matrix metalloproteinase-9. In one embodiment, the amino acid hydroxamic acid derivative is capable of inhibiting gene and protein expressions of matrix metalloproteinase-2 and matrix metalloproteinase-9 of a cancer cell.

In addition, the amino acid hydroxamic acid derivative may comprise an L-form or D-form amino acid hydroxamic acid derivative.

The amino acid hydroxamic acid derivative having a formula of formula (I) may comprise, but is not limited to, _{L}-Glutamic acid γ-hydroxamate (DH) or _{L}-Aspartic acid β-hydroxamate (CH). The formula of the _{L}-Glutamic acid γ-hydroxamate and the formula of _{L}-Aspartic acid β-hydroxamate are as shown as formula (IV) and formula (V), respectively:

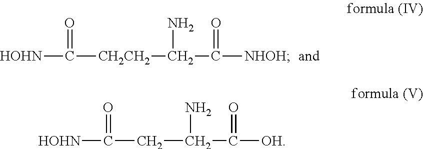

The amino acid hydroxamic acid derivative having a formula of formula (III) may comprise, but is not limited to, _{L}-Glutamic acid dihydroxamate or _{L}-Aspartic acid dihydroxamate. The formula of _{L}-Glutamic acid dihydroxamate and the formula of _{L}-Aspartic acid dihydroxamate are as shown as formula (VI) and formula (VII), respectively:

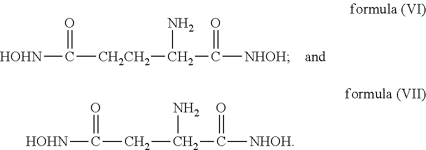

In further another aspect of the invention, the invention may also comprise a method for preparing a cancer metastasis inhibiting drug. The method for preparing a cancer metastasis inhibiting drug comprises providing an effective amount of an amino acid hydroxamic acid derivative in the preparation of the cancer metastasis inhibiting drug, wherein a formula of the amino acid hydroxamic acid derivative is as shown as formula (I), formula (II) or formula (III):

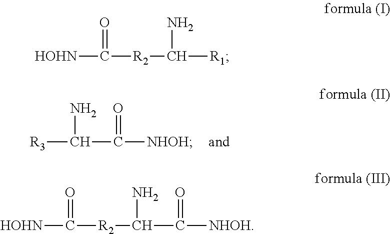

In the above-mentioned formula (I), formula (II) or formula (III), R₁ may comprise carboxyl, C₁-C₆ alkyl, C₁-C₆ alkoxy, NO, or NHOH, R₂ may comprise C₁-C₆ alkyl, C₁-C₆ alkoxy or phenyl, and R₃ may comprise the side chain of tryptophan, the side chain of valine, the side chain of isoleucine, the side chain of threonine, the side chain of lysine, the side chain of phenylalanine, the side chain of leucine, the side chain of methionine, the side chain of histidine, the side chain of glycine, the side chain of glutamic acid, the side chain of hydroxy proline, the side chain of alanine, the side chain of serine, the side chain of glutamine, the side chain of cystine, the side chain of proline, the side chain of aspartic acid, the side chain of citrulline, the side chain of arginine, C₁-C₆ alkyl, C₁-C₆ alkoxy, NO or NHOH.

The above-mentioned amino acid hydroxamic acid derivative may have an effect for inhibiting cancer metastasis. The cancer whose metastasis may be inhibited by the above-mentioned amino acid hydroxamic acid derivative may comprise, but is not limited to, lung cancer, breast cancer, leukaemia, etc. In one embodiment, the effect for inhibiting cancer metastasis may comprise inhibiting cancer cell migration, inhibiting cancer cell invasion, inhibiting cancer cell adhesion and/or inhibiting angiogenesis. The cancer cell may comprise, but is not limited to, a fibrosarcoma cell, a melanoma cell, a breast cancer cell and a lung cancer cell, etc.

The foregoing amino acid hydroxamic acid derivative at a concentration without cytotoxicity may inhibit one of the cancer metastasis biomarkers, matrix metalloproteinase-2 and/or matrix metalloproteinase-9. In one embodiment, the amino acid hydroxamic acid derivative is capable of inhibiting gene and protein expressions of matrix metalloproteinase-2 and matrix metalloproteinase-9 of a cancer cell.

In addition, the amino acid hydroxamic acid derivative may comprise an L-form or D-form amino acid hydroxamic acid derivative.

The amino acid hydroxamic acid derivative having a formula of formula (I) may comprise, but is not limited to, _{L}-Glutamic acid γ-hydroxamate (DH) or _{L}-Aspartic acid β-hydroxamate (CH). The formula of the _{L}-Glutamic acid γ-hydroxamate and the formula of _{L}-Aspartic acid β-hydroxamate are as shown as formula (IV) and formula (V), respectively:

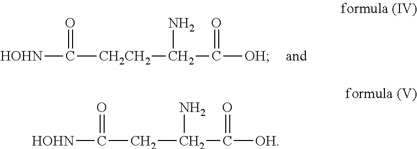

The amino acid hydroxamic acid derivative having a formula of formula (III) may comprise, but is not limited to, _{L}-Glutamic acid dihydroxamate or _{L}-Aspartic acid dihydroxamate. The formula of _{L}-Glutamic acid dihydroxamate and the formula of _{L}-Aspartic acid dihydroxamate are as shown as formula (VI) and formula (VII), respectively:

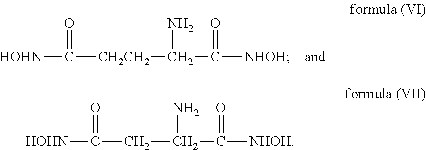

### Example

### Materials and Methods

Materials:

A series of amino acid hydroxamic acid derivatives (_{D, L}-Alanine hydroxamate (AH); _{L}-Arginine hydroxamate hydrochloride (BH); _{L}-Aspartic acid β-hydroxamate (CH); _{L}-Glutamic acid γ-hydroxamate (DH); _{L}-Glycine hydroxamate (EH); _{L}-Lysine hydroxamate (FH), _{D, L}-Serine hydroxamate (GH); _{D, L}-Threonine hydroxamate (HH) (Sigma)), and _{L}-Glutamic acid (D) (Sigma).

Acrylamide (Bio-Rad), Agarose (Bioman), 3-Amino-9-ethylcarbazole (AEC, Sigma), Ammonium persulfate (APS, Merck), Coomassie Blue (Bio-Rad), cAMP Enzyme Immunoassay Kit (Sigma), Dimethyl Sulfoxide (DMSO, J. T. Baker), Dulbecco's Modified Eagle Media (DMEM, Gibco), Matrigel™ Matrix (BD Bioscience), Ethanol (Sigma), Ether (Sigma), Ethylenediaminetetraacetic acid (EDTA, Merck), Fetal Bovine Sera (FBS, Gibco), Enothelial cell growth supplement (Sigma), Formaldehyde (Sigma), Heparin (Sigma), Gelatin (Merck), Hydrogen Peroxide (H₂O₂, Sigma), Isopropanol (Sigma), Methanol (Sigma), MTT (Sigma), Phorbol 12-myristae β-acetate (Sigma), Phosphate Buffer (pH 7.9, Merck), Phosphate-Buffered Saline (Sigma), RIPA Buffer (Sigma), Sodium Bicarbonate (NaHCO₃, Sigma), Sodium Chloride (NaCl, Merck), Sodium Dibasic Phosphate (Na₂HPO₄, Merck), Sodium Hydroxide (NaOH, Merck), Sodium Monobasic Phosphate (NaH₂PO₄, Merck), Toluidine Blue (Sigma), Tris (Merck), Tris-HCl Buffer (pH 7.9, Merck), TRIzol reagent (Sigma), Trypsin-EDTA (0.05% Trypsin with EDTA 4Na, Gibco), Thrombin (Sigma), Tween 20 (Bioman), Tyrosinase (from mushroom, Sigma), Zinc chloride (ZnCl, Sigma), Anti-MMP-2 (Rabbit polyclonal, Sigma) Anti-MMP-9 (Rabbit polyclonal, Sigma), Goat Anti-Rabbit IgG, HRP conjugate (Sigma), Rabbit Anti-Goat IgG, HRP conjugate (Sigma); MMP-9 forward primer 5′-TGGGCTACGTGACCTATGACAT-3′ (SEQ ID. NO.: 1) and reverse primer 5′-GCCCAGCCCACCTCCACTCCTC-3′ (SEQ ID. NO.: 2); MMP-2 forward primer 5′-CAGGCTCTTCTCCTTTCACAA-3′ (SEQ ID. NO.: 3) and reverse primer 5′-AAGCCACGGCTTGGTTTTCCTC-3′ (SEQ ID. NO.: 4); GADPH forward primer 5′-GAGGGGCCATCCACAGTCTTC-3′ (SEQ ID. NO.: 5) and reverse primer 5′-CATCACCATCTTCCAGGAGCG-3′ (SEQ ID. NO.: 6).

Cell Lines:

HT 1080 (Human fibrosarcoma), B16-F10 (Mouse skin melanoma) and Human Umbilical Vein Endothelial Cells (HUVEC) (BCRC No. H-UV001; Bioresource Collection and Research Center (BCRC), Food Industry Research and Development Institute, Taiwan)

Methods:

1. Culture of HT1080 and B16-F10

Upper medium of a culturing plate containing attached cells therein was removed. The cells were washed with a PBS buffer twice, and then the PBS buffer was removed. 5 ml of 0.05% Trypsin-EDTA buffer was added to the culturing plate and shacked slightly to cover the whole bottom of the culturing plate and left still for several minutes. After the Trypsin-EDTA buffer was removed, the cells were observed to see if they were floating and in spherical forms by an inverted microscope. If the cells were floating and in spherical forms, the cells were suspended with 10 ml of DMEM medium containing 10% FBS. After being mixed well, the cells were transferred into a new plate by an appropriate diluting ratio, and then the new plate was placed in an incubator at 37° C. in a humidified atmosphere containing 5% CO₂.

2. Culture of Human Umbilical Vein Endothelial Cells (HUVEC)

An upper medium of a 75T flask containing attached cells therein was removed. The cells were washed with a PBS buffer twice, and then the PBS buffer was removed. 5 ml of 0.05% Trypsin-EDTA buffer was added to the culturing plate and shacked slightly to cover the whole bottom of the 75T flask and left still for several minutes. After the Trypsin-EDTA buffer was removed, the cells were observed to see if they were floating and in spherical forms by an inverted microscope. If the cells were floating and in spherical forms, the cells were suspended with 10 ml of a culturing medium containing 90% M199 (250 unit/ml heparin+3 mg/ml ECGS) and 10% FBS. After being mixed well, the cells with an appropriate diluting ratio was transferred into a new 75T flask, and then the new 75T flask was placed in an incubator at 37° C. in a humidified atmosphere containing 5% CO₂.

3. Cancer Cell Survival Rate Test (Lambert et al., 1997; Yang et al., 2006)

A medium in a culturing plate containing cells grown covering the whole bottom of the culturing plate, was removed. The cells were washed with 10 ml of PBS, and then suspended and mixed well with 10 ml of medium (a part of the cells need to be suspended with Trypsin-EDTA buffer). The numbers of the cells were calculated by a hemocytometer, and the cells were diluted to an appropriate cell concentration and added into the wells of a 24 well culturing plate by an amount of 500 μl/well. The 24 well culturing plates were placed in an incubator at 37° C. in a humidified atmosphere containing 5% CO₂. After the cells were attached onto the bottom of the wells, the medium in each well was removed, and fresh mediums with different concentrations of a test drug were added into wells, respectively (if the cells were suspension cells, the fresh medium may be added into the wells, directly). Then, the 24 well culturing plates were placed in an incubator at 37° C. in a humidified atmosphere containing 5% CO₂ for 24 hours. After that, an upper medium in each well was removed. The cells were washed with a PBS buffer twice, and then an MTT solution which was prepared with a medium was added to each well. After the 24 well culturing plates were placed in an incubator at 37° C. for reacting 4-6 hours, an upper medium in each well was removed, and 100 μl of DMSO was added to each well to solubilize the formazan precipitates. After that, an ELISA reader was used to determine the absorbance for each well at 600 nm.

$$Cytotoxic activity ( \% ) = \frac{Control - Sample}{Control - Medium} \times 100 \%$$

4. Gelatin Gel Zymography (Shim et al., 2003; Park et al., 2005; Hwang et al., 2006)

HT 1080 cells with appropriate cell numbers were cultured in 60 mm culturing plates for 12 hours, respectively. After the cells were attached onto the bottom of the plate and formed a monolayer, the medium in the plate was removed. Fresh serum free mediums with different concentrations of a test sample were added to the plates, respectively, and 100 nM PMA was added to each plate to induce the cells to express great quantities of matrix metalloproteinase-2 and matrix metalloproteinase-9, and the plates were placed in a temperature of 37° C. for 24 hours. After centrifugation, each cultured supernatant were saved and mixed with SDS-sample buffer under the same protein contents which were then injected into a 10% SDS-polyacrylamide gel (containing 1 mg/ml gelatin) and then electrophoresed. After the electrophoresis was completed, the gel was taken out and washed with 20 mM Tris-HCl containing 25% isopropanol three times; 10 minutes per time. Then the gel was equilibrated with 20 mM Tris-HCl for 30 minutes, and finally an incubate buffer (5 mM CaCl₂ and 1 mM ZnCl₂) was added to the gel and reacted with the gel overnight. Then the gel was stained with coomassie blue, and positions having matrix metalloproteinase activity of the gel presented a white color.

5. Protein Quantitative Analysis

A Bradford reagent was diluted with D.I. water by a ratio of 1:4. The 10 μl of a sample solution and 200 μl of a diluted Bradford reagent were added into a 96 well plate and mixed well, and a series of dilutions of the BSA standard solution was used to plot a standard cure. An ELISA reader was used to determine the absorbance at 595 nm for each well. An interpolation method was used to determine the protein content of the sample solution.

6. Western Blot Analysis

HT 1080 cells with appropriate cell numbers were cultured in 60 mm culturing plates for 12 hours, respectively. After the cells were attached onto the bottom of the plate and formed a monolayer, the medium in the plate was removed. Fresh serum-free mediums with different concentrations of a test drug were added to the plates, respectively, and 100 nM PMA was added to each plate to induce the cells to express great quantities of matrix metalloproteinase-2 and matrix metalloproteinase-9, and the plates were placed in a temperature of 37° C. for 24 hours. A quantitatively cultured supernatant in each plate was centrifuged or protein was extracted from cells in each plate to be quantitated, and then an SDS-sample buffer containing 2-ME was added therein to form a mixture. The mixture was injected into a 10% SDS-polyacrylamide gel (containing 1 mg/ml gelatin) and then electrophoresed. After electrophoresis was completed, the proteins in the gel were transferred to a PVDF membrane. The PVDF membrane was equilibrated with gelatin-NET at room temperature for 30 minutes. A primary antibody (dissolved in the gelatin-NET by an appropriate concentration) (anti-metalloproteinase-2 and anti-matrix metalloproteinase-9) was added to the PVDF membrane and reacted with the PVDF membrane at room temperature for 1 hour, and the PVDF membrane was washed with a PBST buffer for three times for 10 minutes each time. A secondary antibody (dissolved in the gelatin-NET by an appropriate concentration) was added to the PVDF membrane and reacted with the PVDF membrane at room temperature for 1 hour, and the PVDF membrane was washed with a PBST buffer for three times for 10 minutes each time. A colorimetric reaction was performed to the PVDF with 10 mM AEC containing 0.03% H₂O₂.

7. RNA Extraction and Reverse Transcription-Polymerase Chain Reaction (RT-PCR)

HT 1080 cells with appropriate cell numbers were cultured in 60 mm culturing plates for 6 hours, respectively. After the cells were attached onto the bottom of the plate and formed a monolayer, the medium in the plate was removed. Fresh serum free mediums with different concentrations of a test sample were added to the plates, respectively, and 100 nM PMA was added to each plate to induce the cells to express great quantities of matrix metalloproteinase-2 and matrix metalloproteinase-9, and the plates were placed in a temperature of 37° C. for 6 hours. After that, the total RNA of the cells were extracted with the TRIzol reagent and reverse transcripted by a reverse transcriptase to synthesize cDNA. The synthesize cDNA was used as a template to perform GADPH, matrix metalloproteinase-2 and matrix metalloproteinase-9 PCR amplifications.

8. Wound Healing Assay (Park et al., 2005; Hwang et al., 2006)

Cells with appropriate cell numbers were cultured in a 24 well culturing plate for 12 hours. After the cells were attached to each bottom of each well of the plate and formed a monolayer, the medium in the well was removed. The middle of each bottom of each well having the monolayer thereon was scraped by a 200 μl tip to form a scratched line with a width of 1 mm. The cells in each well were washed with a PBS buffer twice, and fresh mediums with different concentrations of a test drug were added to the wells, respectively and cultured for 18 hours. After that, the upper medium in each well was removed. The cells were washed with a PBS buffer twice, fixed with 100% methanol and stained with a toluidine blue. The calculation for the migration rate of the cells was:

$$Migration Rate ( \% ) = \frac{Migration distances of drug treated cells}{Migration distances of untreated cells} \times 100 \%$$

9. Colony Dispersion Assay (Hwang et al., 2006)

Cells with appropriate cell numbers were cultured in a 24 well culturing plate for 12 hours. After the cells were attached to each bottom of each well of the plate and formed a monolayer, the medium in the well was removed. Fresh mediums with different concentrations of a test drug were added to the wells, respectively and cultured for 3 days. After that, the upper medium in each well was removed. Then, the cells were washed with a PBS buffer twice, fixed with 100% methanol and stained with a toluidine blue.

10. Invasion Assay (Lee et al., 2005; Huang et al., 2005; Park et al., 2005)

HT 1080 cells were used to prepare a cell suspension with an appropriate concentration by a fresh serum free medium and than placed in each upper chamber of each transwell (BD Matrigel™ Invasion Chamber 24-Well Plate 8.0 Micron). Fresh serum containing mediums with different concentrations of a test drug were added to the lower chambers of the transwells, respectively. The transwells were placed in an incubator containing 5% CO₂ at 37° C. for 18 hours. After that, the medium in the upper chamber was removed and cells on the upper surface of the membrane of the transwell were wiped by a cotton swab. Finally, the cells were fixed and stained. The calculation for the invasion rate of the cells was:

$$InvasionRate ( \% ) = \frac{Nc - Ne}{Nc} \times 100 \%$$

Ne: Numbers of cells crossing the membrane
Nc: Numbers of total cells

11. Cell Adhesion Assay (Lee et al., 2005; Ouyang et al., 2005; Zhang et al., 2005)

HT 1080 cells were used to prepare a cell suspension with an appropriate concentration by a fresh serum free medium, and the cell suspension was mixed with test drugs with different concentrations, respectively and then added to wells of a 24 well culturing plate pre-coated with gelatin, respectively. The 24 well culturing plates were placed in an incubator at 37° C. for 30 minutes. After that, the upper medium in each well was removed and the cells were washed with a PBS buffer twice, and then an MTT solution which was prepared with a medium was added to each well. After the 24 well culturing plates were placed in an incubator at 37° C. for reacting for 4-6 hours, an upper medium in each well was removed, and 100 μl of DMSO was added to each well to solubilize the formazan precipitates. After that, an ELISA reader was used to determine the absorbance at 600 nm for each well. The calculation for the adhesion rate of the cells was:

$$Adhesion Rate ( \% ) = \frac{Blank - Sample}{Blank} \times 100 \%$$

12. Tumor Metastasis in an Animal Model (Itoh et al., 2005; Lee et al., 2005; Ouyang et al., 2005; Zhang et al., 2005)

Experimental animal: C57BL/6, gray-brown fur

Experimental procedure:

C57BL/6 mice were injected with 2×10⁵ cells/0.1 ml of a B16-F10 cell line by a tail intravenous injection. Then, on the next day the mice were injected with a physiological saline solution or 50 mg/kg drug, daily, for 3 weeks. The mice were sacrificed and their lungs were removed at the third week. The lungs were washed with a PBS buffer and fixed with Bouin's solution. The amount and results of metastasize pulmonary nodules were observed for each lung.

13. Tube Formation Assay (Malinda et al., 1999)

1μ-Slide Angiogenesis ibiTreat chambers were coated with 10 μl Matrigel™ Mixtrix and placed in an incubator at 37° C. for 1 hour. 40 μl of a 3×10⁵ cells/ml cell suspension was added to each chamber and left still for 20 minutes to let the cells attach to the chamber. After that, the medium in each chamber was removed. Fresh mediums with different concentrations of a test drug were added to the chambers, respectively and placed in an incubator at 37° C. After 18 hours, the angiogenesis appearance in each chamber was observed and photographed for recording.

Results:

1. Analysis of Cytotoxicity of Amino Acid Hydroxamic Acid Derivatives Matrix Metalloproteinase-2 and Matrix Metalloproteinase-9 of a Cancer Cell to HT 1080 Cells

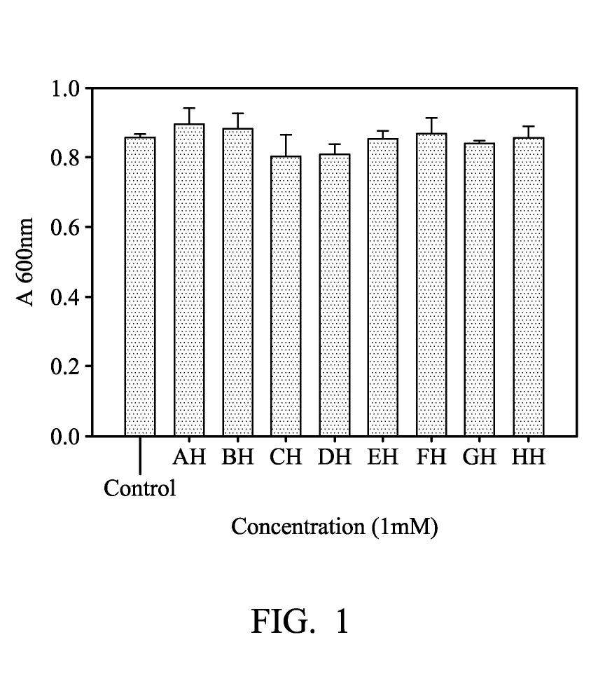

In the experiment, selection was performed to 8 kinds of amino acid hydroxamic acid derivatives by their abilities for inhibiting activity of the matrix metalloproteinase produced from HT 1080 cells. 24 hours after the cell line was added to each kind of the amino acid hydroxamic acid derivatives, respectively, cell survival rate for the cell line added to each kind of the amino acid hydroxamic acid derivatives was analyzed by using an MTT, and the results are shown as FIG. 1. Referring to FIG. 1, for 1 a mM concentration of all test drugs, when compared to the control group, cytotoxicities of the 8 kinds of amino acid hydroxamic acid derivatives, AH to HH, to HT 1080 cells and that of the control group had no significant difference. It was shown that the 8 kinds of the amino acid hydroxamic acid derivatives at the concentrations mentioned above have almost no cytotoxicity.

2. Determination of Matrix Metalloproteinase-2 and Matrix Metalloproteinase-9 of a Cancer Cell Inhibiting Activities of Matrix Metalloproteinase-2 and Matrix Metalloproteinase-9 of a Cancer Cell

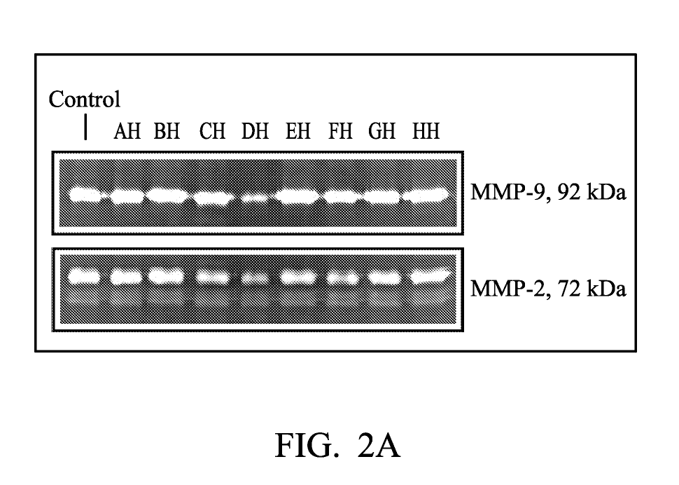

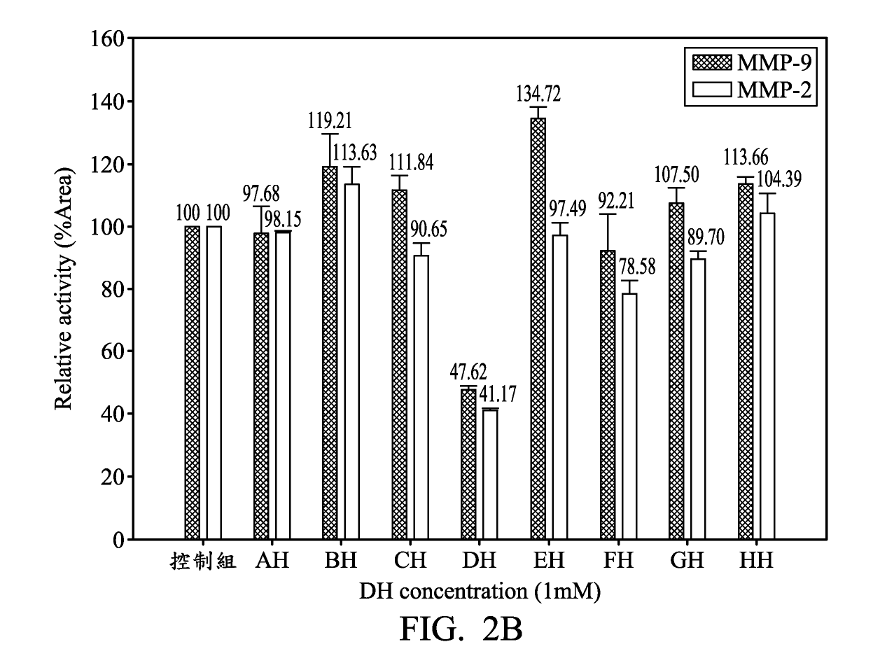

HT 1080 cells were treated with 8 kinds of the amino acid hydroxamic acid derivatives (AH to HH) for 24 hours, respectively, and the effects of the 8 kinds of the amino acid hydroxamic acid derivatives to the matrix metalloproteinase-2 and matrix metalloproteinase-9 were observed, respectively. The results are shown as FIGS. 2 (A) and 2 (B). The results show that the _{L}-Glutamic acid γ-hydroxamate (DH) caused the activities of matrix metalloproteinase-2 and matrix metalloproteinase-9 to most significantly decrease. For the _{L}-Glutamic acid γ-hydroxamate (DH), the inhibition rate to the activities of matrix metalloproteinase-2 and matrix metalloproteinase-9 were greater than 50%.

3. Cytotoxicity Tests for _{L}-Glutamic Acid γ-Hydroxamate (DH) and the Structural Control Compound Thereof, _{L}-Glutamic Acid (D), to HT 1080 Cells

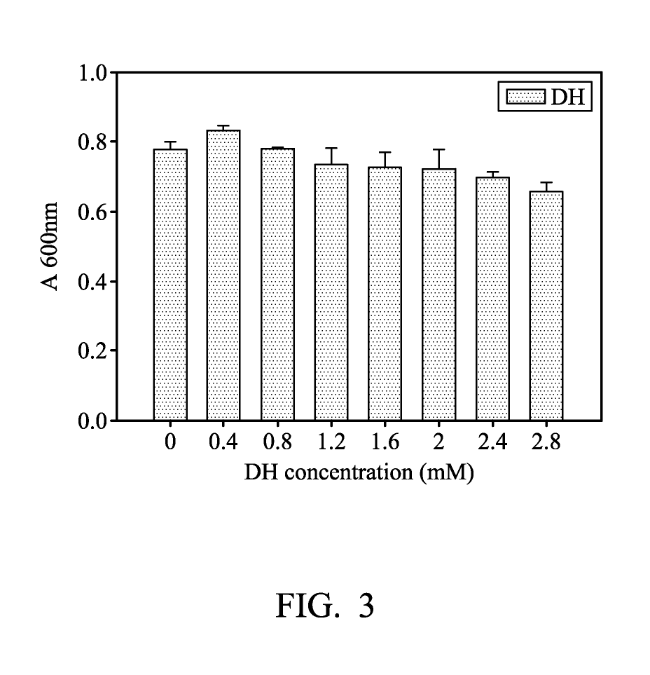

HT 1080 cells were treated with _{L}-Glutamic acid γ-hydroxamate (DH) and _{L}-Glutamic acid (D) for 24 hours, respectively, and the cell viability of cells treated with _{L}-Glutamic acid γ-hydroxamate (DH) and cells treated with _{L}-Glutamic acid (D) were observed. The results are shown as FIG. 3. According to FIG. 3, it is shown that _{L}-Glutamic acid γ-hydroxamate (DH) had no significant cytotoxicity to HT 1080 cells.

4. Inhibition Tests for _{L}-Glutamic Acid γ-Hydroxamate (DH) and the Structural Control Compound Thereof, _{L}-Glutamic Acid (D), to Activities of Matrix Metalloproteinase-2 and Matrix Metalloproteinase-9 of Cancer Cells

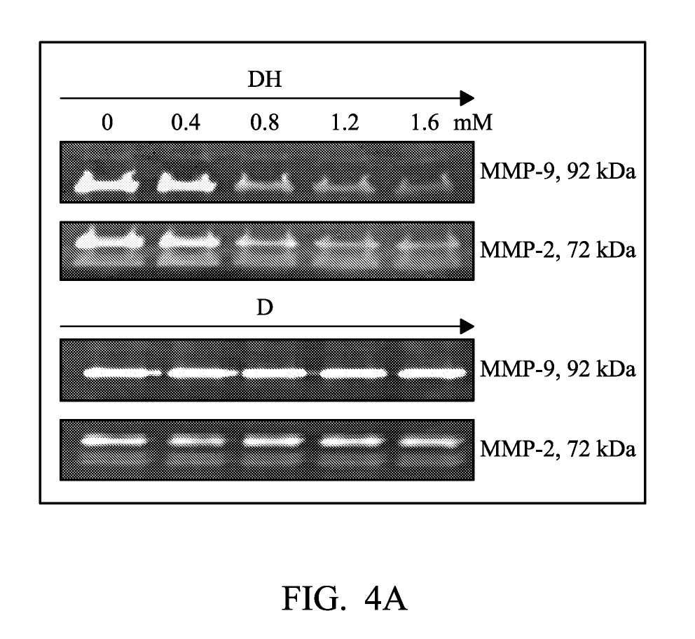

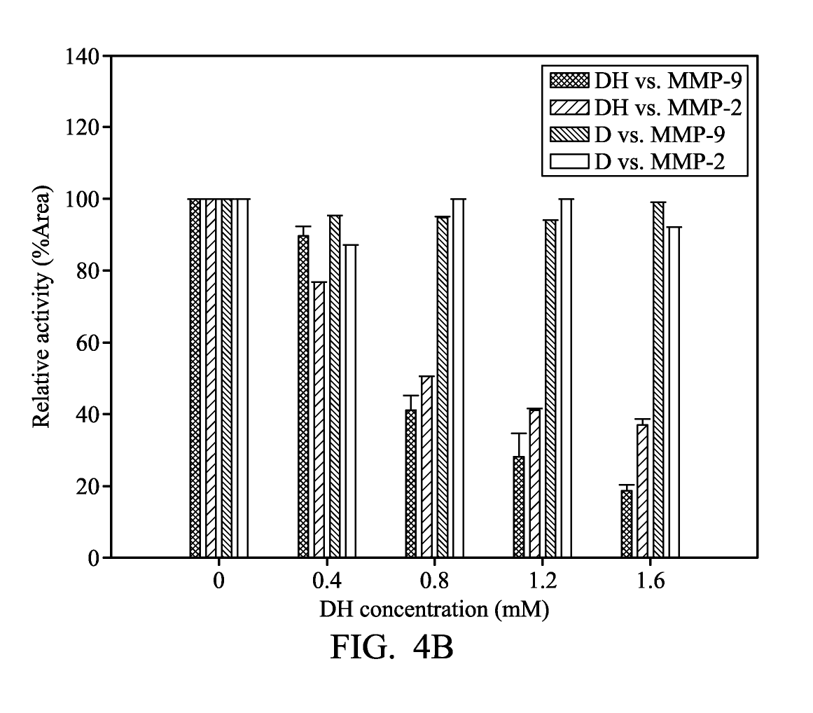

HT 1080 cells were treated with the different concentrations of _{L}-Glutamic acid γ-hydroxamate (DH) and different concentrations of _{L}-Glutamic acid (D) for 24 hours, respectively. After that MTT analyses was performed to the cells treated with the different concentrations of _{L}-Glutamic acid γ-hydroxamate (DH) and cells treated with the different concentrations of _{L}-Glutamic acid (D), to know the effects of different concentrations of _{L}-Glutamic acid γ-hydroxamate (DH) and different concentrations of _{L}-Glutamic acid (D) on the matrix metalloproteinase-2 and matrix metalloproteinase-9. The results are shown as FIGS. 4 (A) and 4 (B). According to the matrix metalloproteinase-2 and matrix metalloproteinase-9 inhibiting activities of _{L}-Glutamic acid γ-hydroxamate (DH) at concentrations of 0.4, 0.8, 1.2 and 1.6 mM shown in FIGS. 4 (A) and 4(B), it was shown that the matrix metalloproteinase-2 and matrix metalloproteinase-9 inhibiting activities of _{L}-Glutamic acid γ-hydroxamate (DH) was increased while the concentration thereof was increased. However, _{L}-Glutamic acid (D) had no inhibiting effect on the matrix metalloproteinase-2 and matrix metalloproteinase-9.

5. Extracellular Inhibition Tests for _{L}-Glutamic Acid γ-Hydroxamate (DH) to Enzyme Activities of Matrix Metalloproteinase-2 and Matrix Metalloproteinase-9

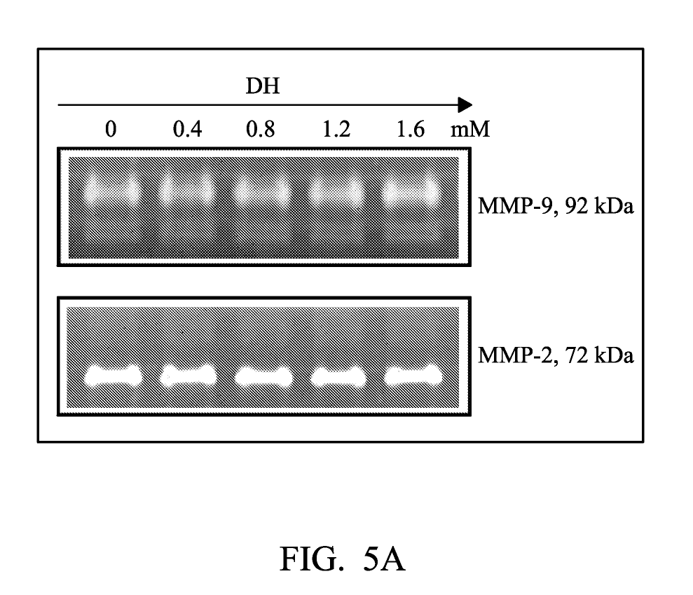

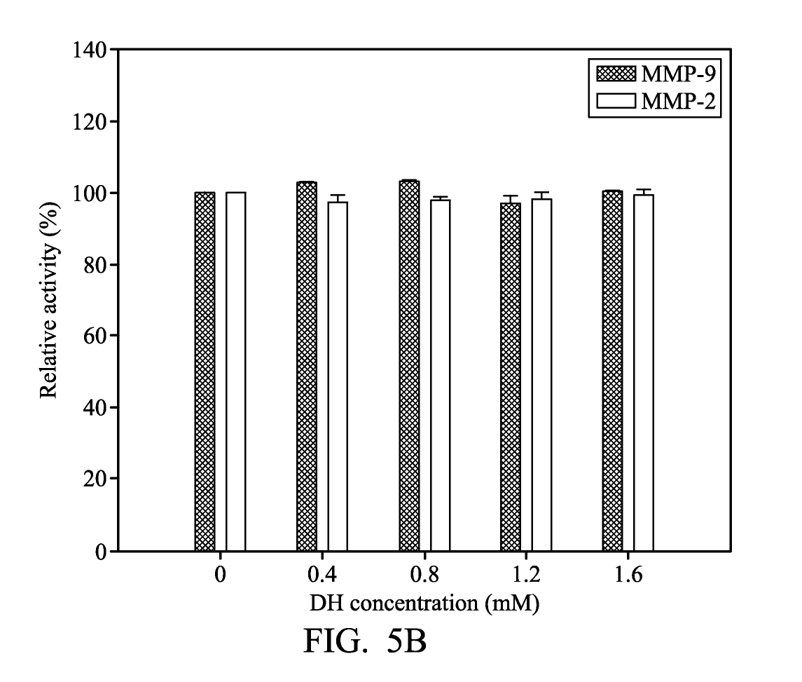

HT 1080 cells were induced with PMA, and the cultured medium for culturing the HT 1080 cells therefrom was taken out and treated with the different concentrations of _{L}-Glutamic acid γ-hydroxamate (DH) for 24 hours, respectively. After that the enzyme activities of the matrix metalloproteinase-2 and matrix metalloproteinase-9 in the medium were analyzed. The results are shown as FIGS. 5(A) and 5(B). According to the results in FIGS. 4 (A) and 4 (B), it was shown that _{L}-Glutamic acid γ-hydroxamate (DH) had an inhibiting effect on the matrix metalloproteinase-2 and matrix metalloproteinase-9 while _{L}-Glutamic acid (D) had no inhibiting effect to the matrix metalloproteinase-2 and matrix metalloproteinase-9. Therefore, whether the inhibition effect for _{L}-Glutamic acid γ-hydroxamate (DH) to the matrix metalloproteinase-2 and matrix metalloproteinase-9 is related to the direct inhibition of the enzyme activities of matrix metalloproteinase-2 and matrix metalloproteinase-9 was investigated. In FIGS. 5 (A) and 5 (B), it was found that after the matrix metalloproteinase-2 and matrix metalloproteinase-9 were secreted from the PMA induced HT 1080 was reacted with different concentrations of _{L}-Glutamic acid γ-hydroxamate (DH) for 24 hours, respectively, there was no inhibition to the enzyme activities of the matrix metalloproteinase-2 and matrix metalloproteinase-9. Therefore, it was known that the inhibiting by the _{L}-Glutamic acid γ-hydroxamate (DH) of the matrix metalloproteinase-2 and matrix metalloproteinase-9 is not due to direct inhibition of the enzyme activities of the matrix metalloproteinase-2 and matrix metalloproteinase-9.

6. Inhibition Tests for _{L}-Glutamic Acid γ-Hydroxamate (DH) to Protein Expressions of Matrix Metalloproteinase-2 and Matrix Metalloproteinase-9 of Cancer Cells

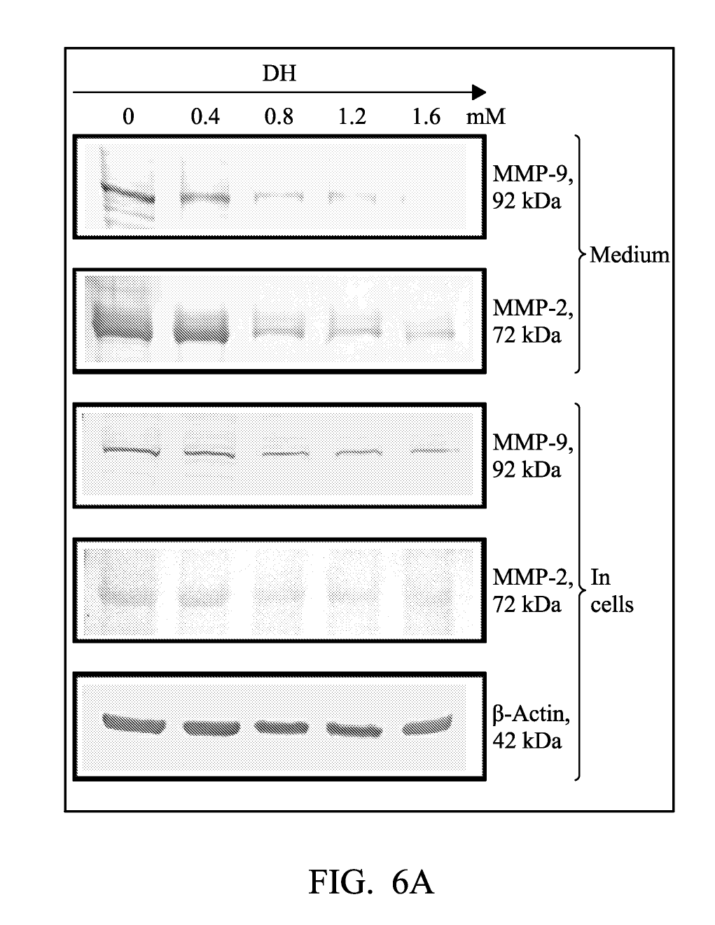

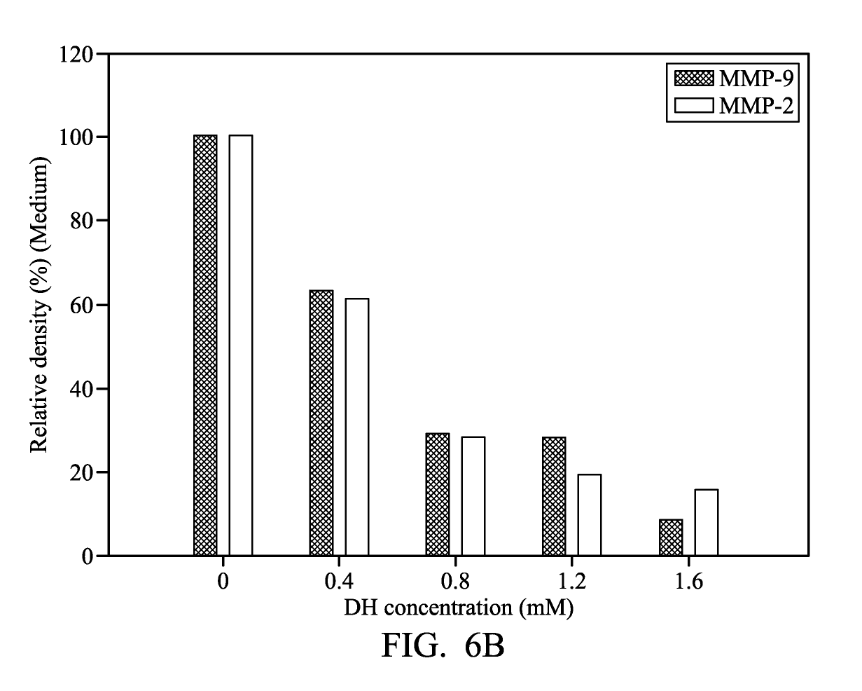

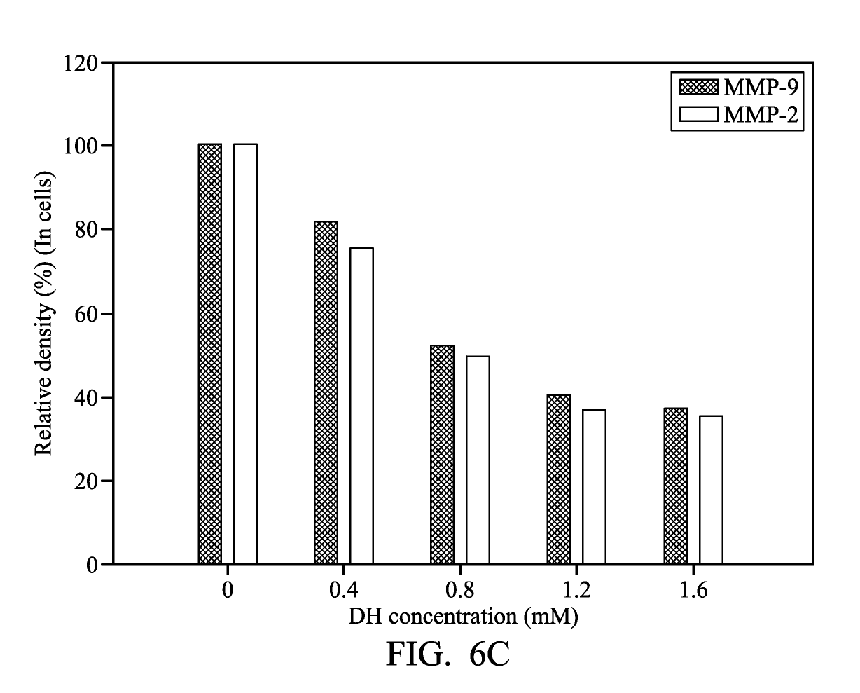

After the HT 1080 cells were treated with the different concentrations of _{L}-Glutamic acid γ-hydroxamate (DH) for 24 hours, respectively, the protein expression amounts and protein activities of the matrix metalloproteinase-2 and matrix metalloproteinase-9 secreting out the cells and left in the cells were observed. The results are shown as FIG. 6 (A) and FIGS. 6 (C) and (B), respectively.

In FIG. 6 (A), by using Western blotting, it was known that after the HT 1080 cells were treated with _{L}-Glutamic acid γ-hydroxamate (DH) at a concentration of 0.4, 0.8, 1.2, and 1.6 for 24 hours, respectively, the protein expression amounts of matrix metalloproteinase-2 and matrix metalloproteinase-9 secreting out of the HT 1080 cells and left in the HT 1080 cells were all decreased, and the decreasing trend was related to the concentrations of _{L}-Glutamic acid γ-hydroxamate (DH). FIGS. 6 (B) and 6 (C) showed that since the protein expression amounts of matrix metalloproteinase-2 and matrix metalloproteinase-9 secreting out of the HT 1080 cells and left in the HT 1080 cells all decreased, the protein activities of the matrix metalloproteinase-2 and matrix metalloproteinase-9 secreting out of the HT 1080 cells and left in the HT 1080 cells also decreased.

7. Inhibition Tests for _{L}-Glutamic Acid γ-Hydroxamate (DH) to Gene Expressions of Matrix Metalloproteinase-2 and Matrix Metalloproteinase-9 of Cancer Cells

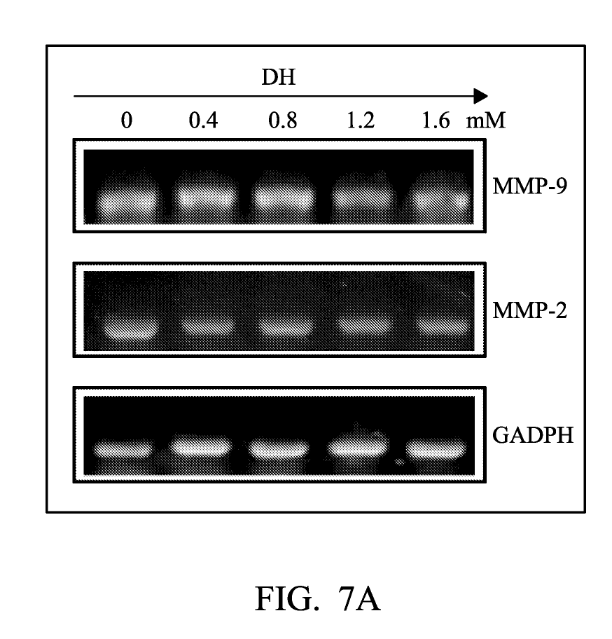

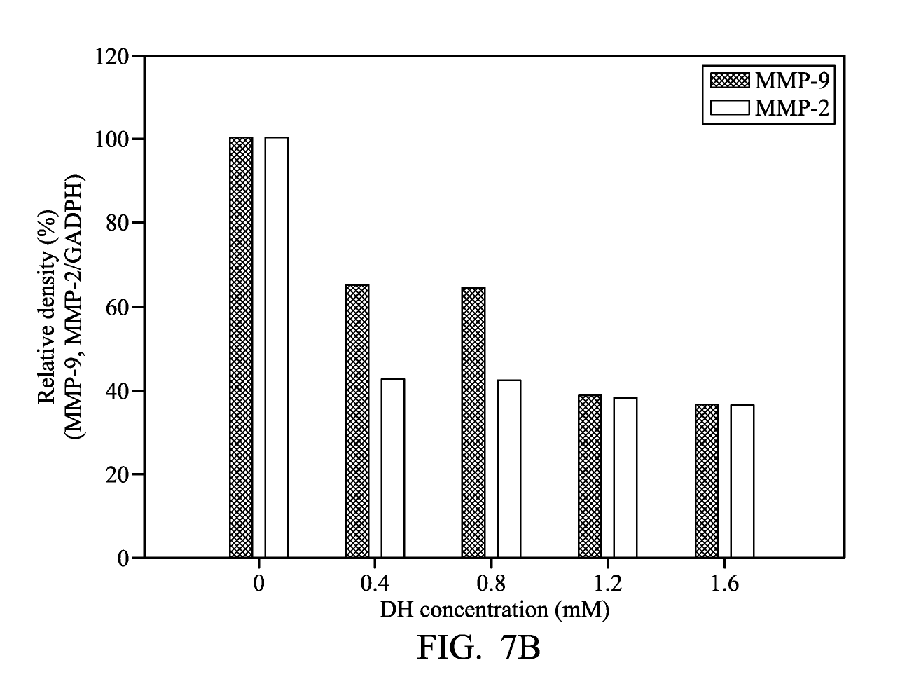

After the HT 1080 cells were treated with the different concentrations of _{L}-Glutamic acid γ-hydroxamate (DH) for 6 hours, respectively, the reverse transcription-polymerase chain reactions were used to analyze the effect of _{L}-Glutamic acid γ-hydroxamate (DH) to the mRNA expressions of matrix metalloproteinase-2 and matrix metalloproteinase-9 of the HT 1080 cells. The results are shown as FIGS. 7 (A) and (B). FIGS. 7 (A) and (B) showed that after the HT 1080 cells were treated with _{L}-Glutamic acid γ-hydroxamate (DH) at a concentration of 0.4, 0.8, 1.2, and 1.6 for 6 hours, respectively, the gene expressions of matrix metalloproteinase-2 and matrix metalloproteinase-9 of the HT 1080 cells decreased, and the decreasing trend was related to the concentration of _{L}-Glutamic acid γ-hydroxamate (DH).

8. Inhibition Effect of _{L}-Glutamic Acid γ-Hydroxamate (DH) to Cell Migration

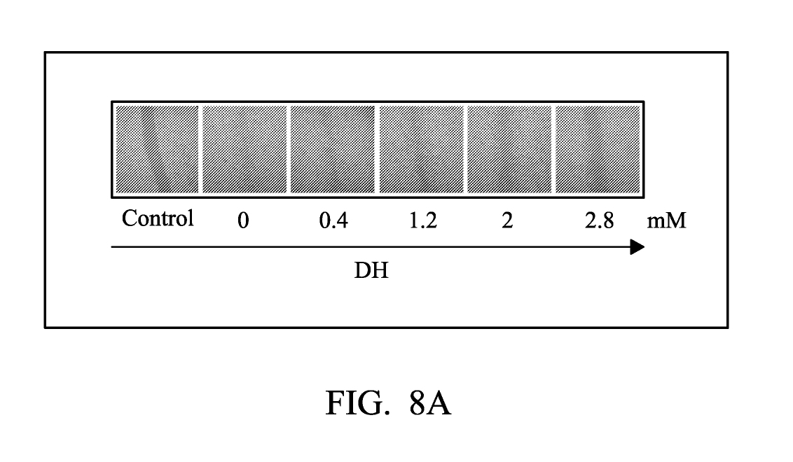

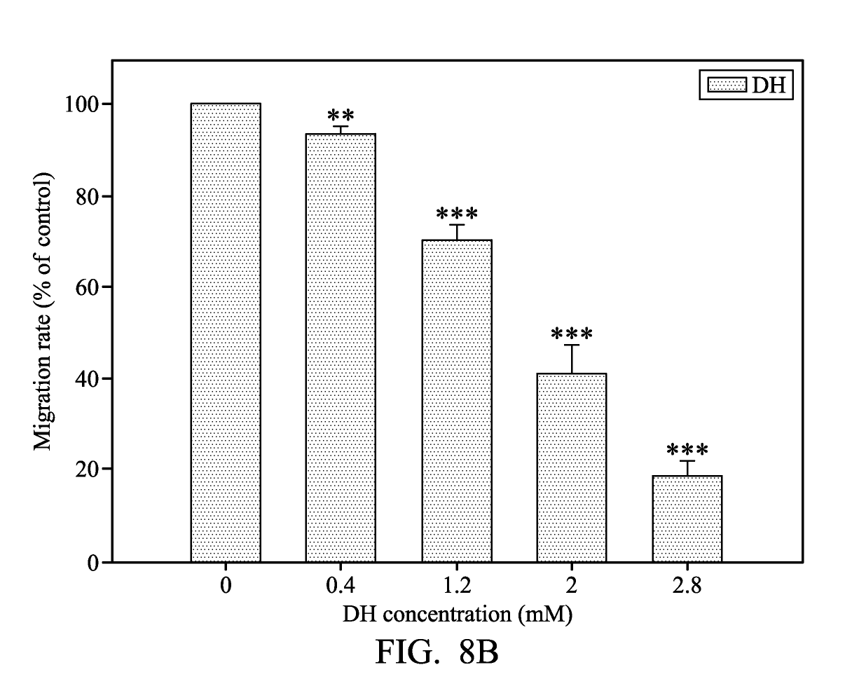

After the HT 1080 cells were treated with _{L}-Glutamic acid γ-hydroxamate (DH) at a concentration of 0.4, 1.2, 2 and 2.8 mM for 18 hours, respectively, the wound healing assay and colony dispersion assay were performed to the treated HT 1080 cells, respectively. The results are shown as FIGS. 8(A) and (B). The results showed that in a condition in which the cell migration of the HT 1080 cells were significantly inhibited by the _{L}-Glutamic acid γ-hydroxamate (DH), IC₅₀ of the _{L}-Glutamic acid γ-hydroxamate (DH) was 1.75 mM.

10. Inhibition Effect of _{L}-Glutamic Acid γ-Hydroxamate (DH) to Cell Migration

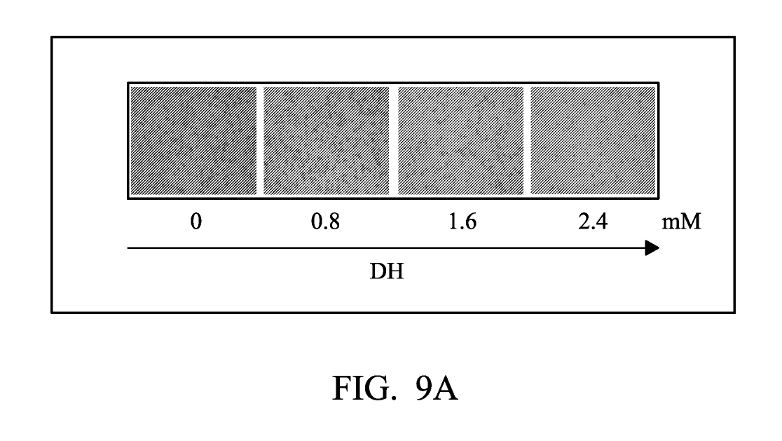

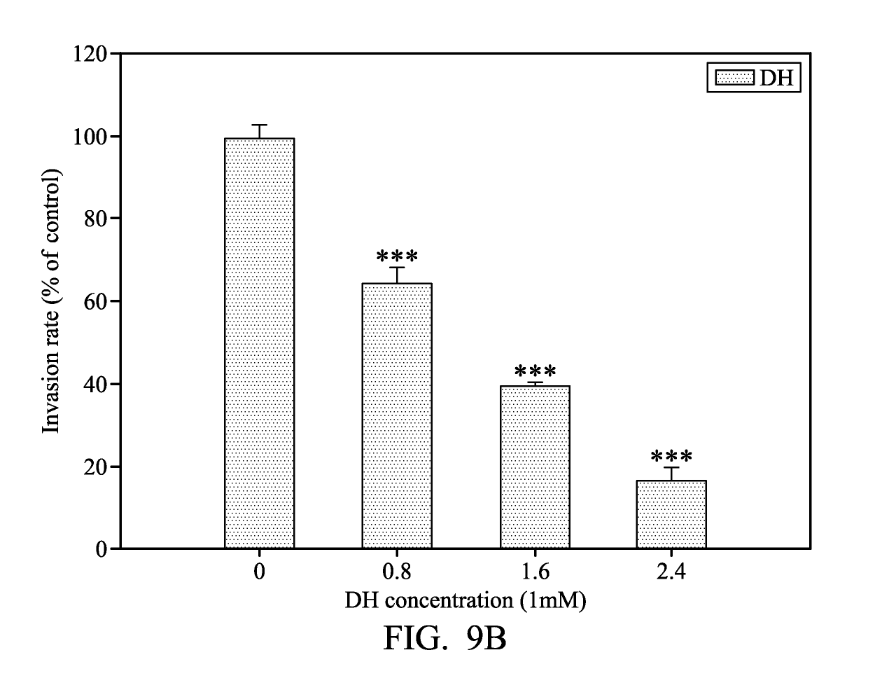

After the HT 1080 cells were placed in upper chambers coated with Martrigeff of the transwells and treated with _{L}-Glutamic acid γ-hydroxamate (DH) at a concentration of 0.8, 1.6 and 2.4 mM for 18 hours, respectively, the inhibition of crossing membrane of the treated HT 1080 cells were observed. The results are shown as FIGS. 9(A) and (B). The results showed that the HT 1080 crossing membrane to the lower chamber of the transwells were significantly inhibited by the _{L}-Glutamic acid γ-hydroxamate (DH), IC₅₀ of the _{L}-Glutamic acid γ-hydroxamate (DH) was 1.262 mM.

11. Inhibition Effect of L-Glutamic Acid γ-Hydroxamate (DH) to Extracellular Matrix Adhesion for HT 1080 Cells

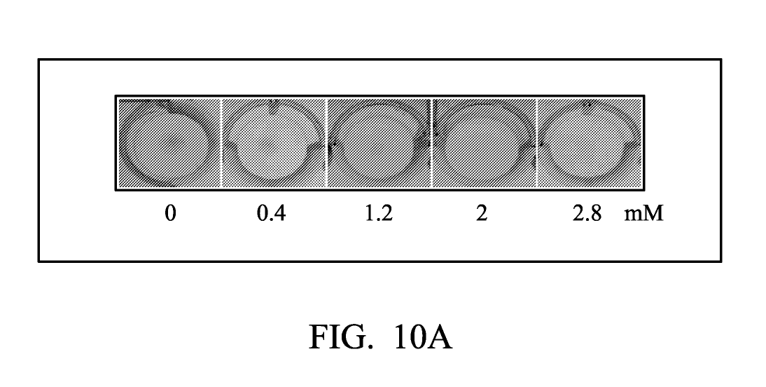

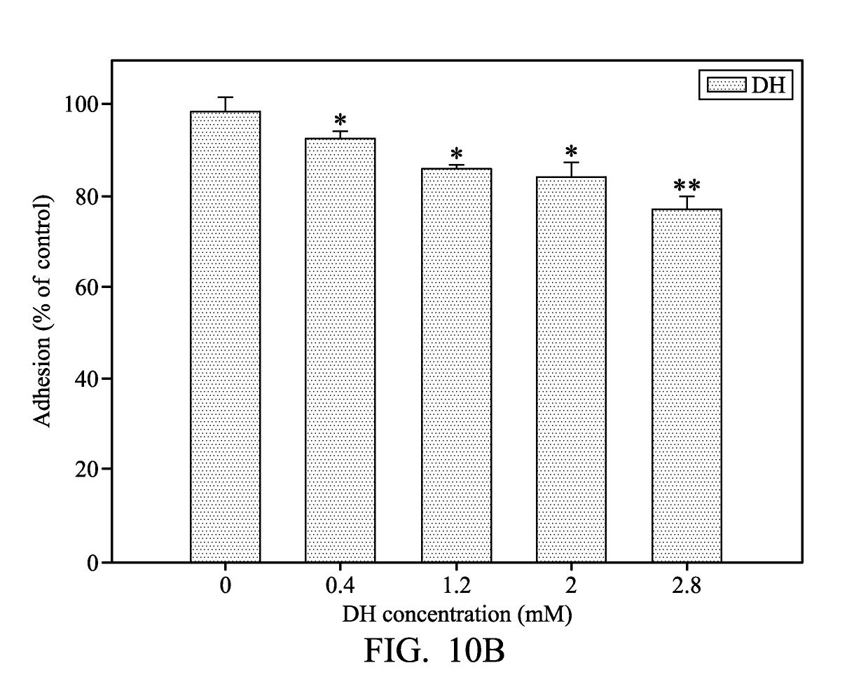

HT 1080 cell suspensions with appropriate cell concentrations were treated with _{L}-Glutamic acid γ-hydroxamate (DH) at a concentration of 0.4, 1.2, 2 and 2.8 mM, and placed in culturing plates coated with gelatin, respectively for reacting for 30 minutes for performing adhesion tests. The results are shown as FIG. 10. The results showed that with concentration of _{L}-Glutamic acid γ-hydroxamate (DH) increased, and the number of the HT 1080 cells attached in the surface of the bottom of the culturing plate presented a slight dose dependent decreasing trend.

12. In Vivo Experiment

(1) Effects of _{L}-Glutamic Acid γ-Hydroxamate (DH) on Lung Metastasis and Viability of Mice

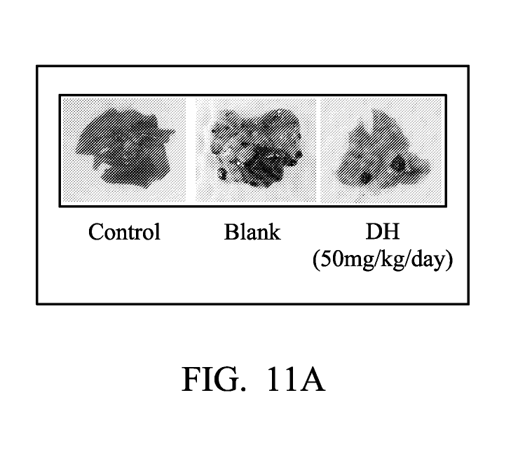

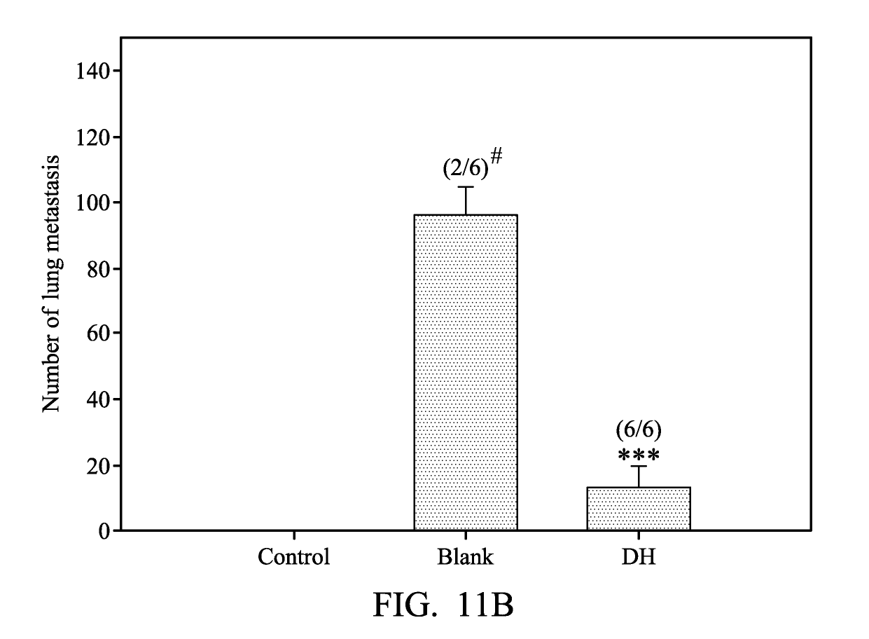

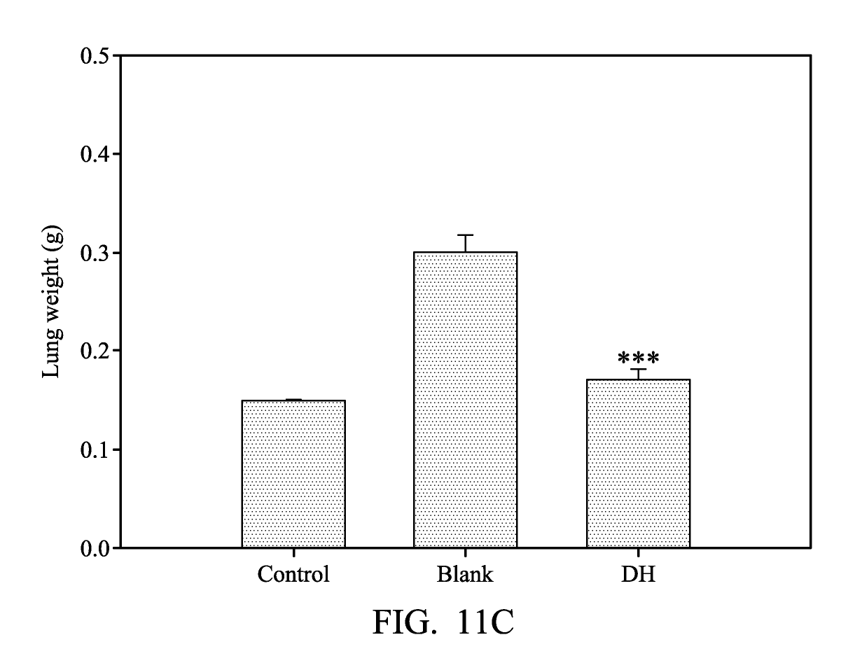

After the effect of the _{L}-Glutamic acid γ-hydroxamate (DH) inhibiting human fibrosarcomas, HT 1080 cells, was confirmed by the in vitro experiments, the in vivo experiments was further performed to observe the effect of the _{L}-Glutamic acid γ-hydroxamate (DH) on an animal model. In the animal model of lung metastasis induced by injecting B16-F10 cell line to mice by a tail intravenous injection, the mice were injected with 50 mg/kg/day _{L}-Glutamic acid γ-hydroxamate (DH), for 21 days. The results are shown in FIGS. 11 (A), (B) and (C). FIGS. 11 (A) and (B) showed that _{L}-Glutamic acid γ-hydroxamate (DH) significantly decreased lung metastasis and the amount of metastasized pulmonary nodules. FIGS. 11 (C) showed that in the drug administered group, _{L}-Glutamic acid γ-hydroxamate (DH) was capable of inhibiting the increase of the lung weight due to cancer metastasis and the lung weight of the mice recovered to a level similar to that of the control group (no cancer induced group). In addition, in the blank group (cancer induced without a drug being administered), only 2 of the 6 mice survived till the end of the experiment, while in the drug administered group, the six mice all survived. Therefore, according to the animal experiment, it is known that _{L}-Glutamic acid γ-hydroxamate (DH) is capable of increasing mice viability

(2) Effects of _{L}-Glutamic Acid γ-Hydroxamate (DH) on Appearances and Weights of the Mice

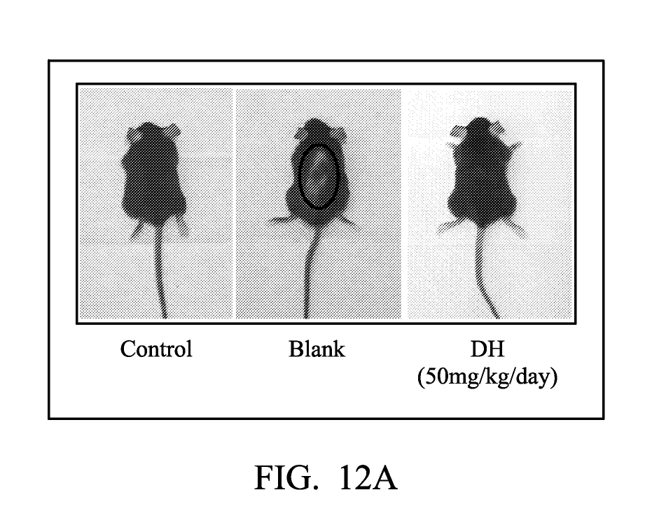

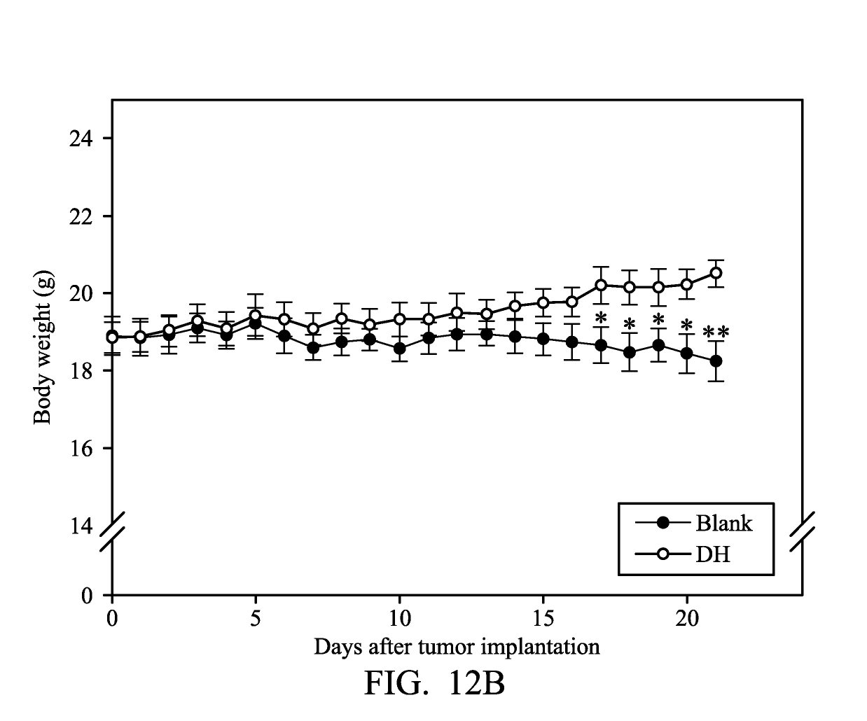

The appearances and weights of the mice of the control group, drug administered group and blank group were observed and recorded, and the results are shown as FIGS. 12 (A) and (B). As compared to the mice of the blank group, for the mice of the drug administered group, the pathological symptoms such as fur picking (FIG. 12 (A), position indicated by thick line circle), weight loss (FIG. 12 (B)), hind limb debility, etc. due to cancer metastasis, decreased.

13. Effects of _{L}-Aspartic Acid β-Hydroxamate (CH) and _{L}-Glutamic Acid γ-Hydroxamate (DH) on Viability of Human Umbilical Vein Endothelial Cells and Angiogenesis

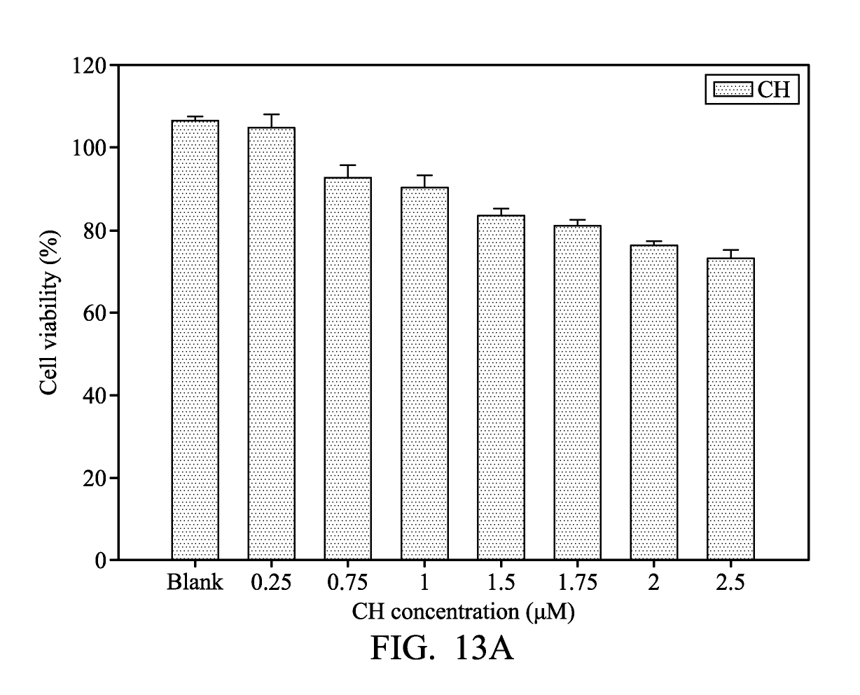

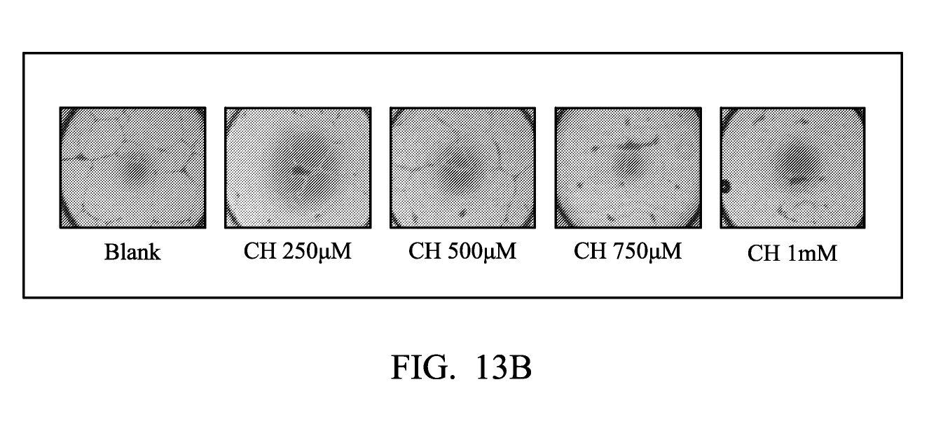

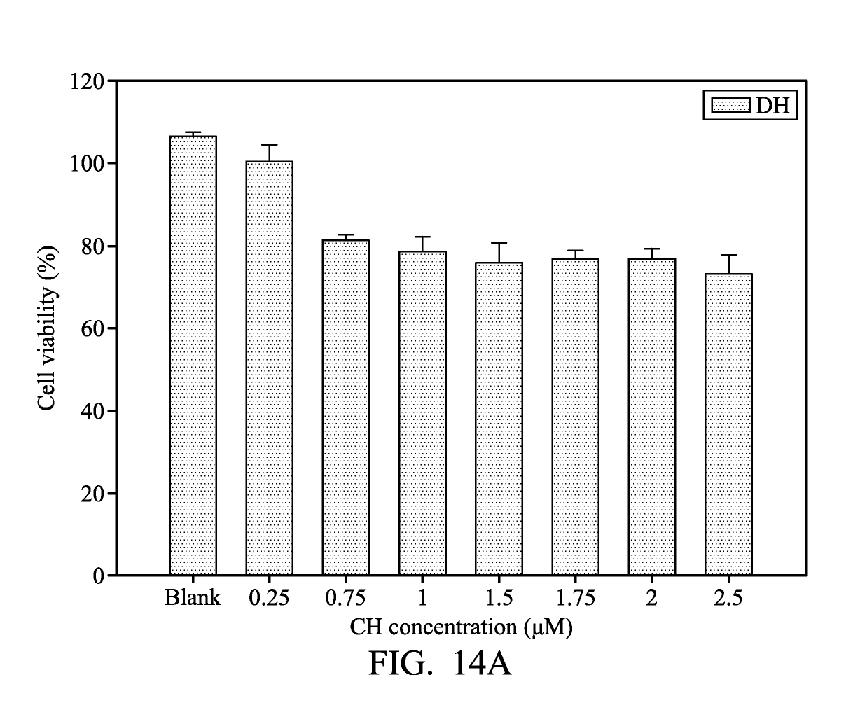

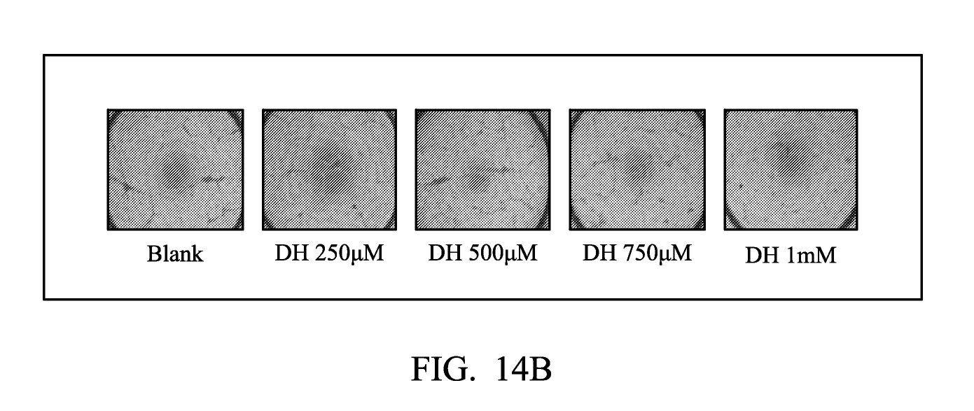

After human umbilical vein endothelial cells were treated with the different concentrations of _{L}-Aspartic acid β-hydroxamate (CH) and different concentrations of _{L}-Glutamic acid γ-hydroxamate (DH) for 24 hours, respectively, the cell viability assays were performed to the treated human umbilical vein endothelial cells and the angiogenesis conditions of the human umbilical vein endothelial cells were recorded. The results of the cell viability assays are shown as FIGS. 13 (A) and 14 (A). Photographs for the human umbilical vein endothelial cells treated with _{L}-Aspartic acid β-hydroxamate (CH) and human umbilical vein endothelial cells treated with _{L}-Glutamic acid γ-hydroxamate (DH) are shown as FIGS. 13 (B) and 14 (B), respectively.

(A) Cell Viability Assays

FIGS. 13 (A) and 14 (A) showed that at the concentration of 0.25 mM, _{L}-Aspartic acid β-hydroxamate (CH) and _{L}-Glutamic acid γ-hydroxamate (DH) both had no cytotoxicity to the human umbilical vein endothelial cells. While the concentrations of _{L}-Aspartic acid β-hydroxamate (CH) and _{L}-Glutamic acid γ-hydroxamate (DH) were raised to 0.75 mM, there were slight decreases in the viability of human umbilical vein endothelial cells.

(B) Angiogenesis

According to FIGS. 13 (B) and 14 (B), it was shown that as compared with the control group, angiogenesis of the _{L}-Aspartic acid β-hydroxamate (CH) treated groups and _{L}-Glutamic acid γ-hydroxamate (DH) treated groups was significantly inhibited with increasing concentrations of _{L}-Aspartic acid β-hydroxamate (CH) and _{L}-Glutamic acid γ-hydroxamate (DH). At the concentration of 0.25 mM, _{L}-Aspartic acid β-hydroxamate (CH) already had an effect of inhibiting angiogenesis, which was better than _{L}-Glutamic acid γ-hydroxamate (DH) at the same concentration.

While the invention has been described by way of example and in terms of the preferred embodiments, it is to be understood that the invention is not limited to the disclosed embodiments. To the contrary, it is intended to cover various modifications and similar arrangements (as would be apparent to those skilled in the art). Therefore, the scope of the appended claims should be accorded the broadest interpretation so as to encompass all such modifications and similar arrangements.

What is claimed is:

1. A composition for inhibiting cancer metastasis, comprising
an effective amount of an amino acid hydroxamic acid derivative having a formula as shown as formula (I), formula (II) or formula (III): 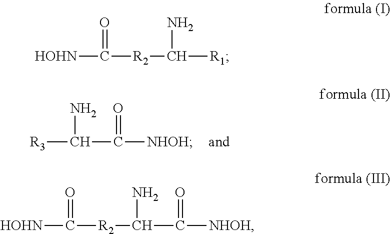
wherein R₁ comprises carboxyl, C₁-C₆ alkyl, C₁-C₆ alkoxy, NO, or NHOH, R₂ comprises C₁-C₆ alkyl, C₁-C₆ alkoxy or phenyl, and R₃ comprises the side chain of tryptophan, the side chain of valine, the side chain of isoleucine, the side chain of threonine, the side chain of lysine, the side chain of phenylalanine, the side chain of leucine, the side chain of methionine, the side chain of histidine, the side chain of glycine, the side chain of glutamic acid, the side chain of hydroxy proline, the side chain of alanine, the side chain of serine, the side chain of glutamine, the side chain of cystine, the side chain of proline, the side chain of aspartic acid, the side chain of citrulline, the side chain of arginine, C₁-C₆ alkyl, C₁-C₆ alkoxy, NO or NHOH; and
a pharmaceutically acceptable carrier or salt,
wherein the amino acid hydroxamic acid derivative has an effect for inhibiting cancer metastasis.

2. The composition for inhibiting cancer metastasis as claimed in claim 1, wherein a content of the amino acid hydroxamic acid derivative is about 0.01-50 wt %.

3. The composition for inhibiting cancer metastasis as claimed in claim 1, wherein the effect for inhibiting cancer metastasis comprises inhibiting cancer cell migration, inhibiting cancer cell invasion, inhibiting cancer cell adhesion and/or inhibiting angiogenesis.

4. The composition for inhibiting cancer metastasis as claimed in claim 1, wherein the amino acid hydroxamic acid derivative is capable of inhibiting gene and protein expressions of matrix metalloproteinase-2 and matrix metalloproteinase-9 of a cancer cell.

5. The composition for inhibiting cancer metastasis as claimed in claim 1, wherein the amino acid hydroxamic acid derivative comprises an L-form or D-form amino acid hydroxamic acid derivative.

6. The composition for inhibiting cancer metastasis as claimed in claim 1, wherein the amino acid hydroxamic acid derivative having a formula of formula (I) comprises _{L}-Glutamic acid γ-hydroxamate (DH) or _{L}-Aspartic acid β-hydroxamate (CH), and the formula of the _{L}-Glutamic acid γ-hydroxamate and the formula of _{L}-Aspartic acid β-hydroxamate are as shown as formula (IV) and formula (V), respectively:

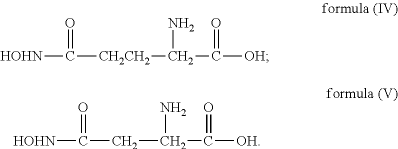

7. The composition for inhibiting cancer metastasis as claimed in claim 1, wherein the amino acid hydroxamic acid derivative having a formula of formula (I) _{L}-Glutamic acid γ-hydroxamate (DH), and the formula of the _{L}-Glutamic acid γ-hydroxamateis is as shown as formula (IV):

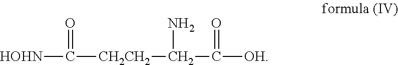

8. The composition for inhibiting cancer metastasis as claimed in claim 7, wherein the effect for inhibiting cancer metastasis comprises inhibiting cancer cell migration, inhibiting cancer cell invasion, inhibiting cancer cell adhesion and/or inhibiting angiogenesis.

9. The composition for inhibiting cancer metastasis as claimed in claim 7, wherein the _{L}-Glutamic acid γ-hydroxamate is capable of inhibiting gene and protein expressions of matrix metalloproteinase-2 and matrix metalloproteinase-9 of a cancer cell.

10. The composition for inhibiting cancer metastasis as claimed in claim 1, wherein the amino acid hydroxamic acid derivative having a formula of formula (III) comprises _{L}-Glutamic acid dihydroxamate or _{L}-Aspartic acid dihydroxamate, and the formula of _{L}-Glutamic acid dihydroxamate and the formula of _{L}-Aspartic acid dihydroxamate are as shown as formula (VI) and formula (VII), respectively: [chemical structure]


SEQUENCE LISTING (6 sequences)

SEQ ID NO: 1 - DNA, 21 bp, Artificial Sequence
  MMP-2 forward primer
caggctcttctcctttcacaa

SEQ ID NO: 2 - DNA, 22 bp, Artificial Sequence
  MMP-2 reverse primer
aagccacggcttggttttcctc

SEQ ID NO: 3 - DNA, 22 bp, Artificial Sequence
  MMP-9 forward primer
tgggctacgtgacctatgacat

SEQ ID NO: 4 - DNA, 22 bp, Artificial Sequence
  MMP-9 reverse primer
gcccagcccacctccactcctc

SEQ ID NO: 5 - DNA, 21 bp, Artificial Sequence
  GADPH forward primer
gaggggccatccacagtcttc

SEQ ID NO: 6 - DNA, 21 bp, Artificial Sequence
  GADPH reverse primer
catcaccatcttccaggagcg